(19) United States
(12) Patent Application Publication
(10) Pub. No.: US 2024/0285190 A1
(43) Pub. Date: Aug. 29, 2024

(54) EAR-WEARABLE SYSTEMS FOR GAIT ANALYSIS AND GAIT TRAINING

(71) Applicant: Starkey Laboratories, Inc., Eden Prairie, MN (US)

(72) Inventors: Achintya Kumar Bhowmik, Cupertino, CA (US); Bernard Bechara, Minneapolis, MN (US); Justin R. Burwinkel, Eden Prairie, MN (US); Krishna Chaithanya Vastare, Eden Prairie, MN (US); Gerard N. Weisensel, Savage, MN (US)

(21) Appl. No.: 18/573,225

(22) PCT Filed: Jun. 21, 2022
(86) PCT No.: PCT/US2022/034287

§ 371 Date: Dec. 21, 2023

Related U.S. Application Data
(60) Provisional application No. 63/212,906, filed on Jun. 21, 2021.

(51) Int. Cl. A61B 5/11; A61B 5/00

(52) U.S. Cl. CPC A61B 5/112; A61B 5/6817; A61B 2505/09

(57) ABSTRACT

Embodiments herein relate to car-wearable devices and systems that can analyze a device wearer's gait and/or provide gait training. In a first aspect, an car-wearable device is included having a control circuit, a motion sensor in electrical communication with the control circuit, a microphone in electrical communication with the control circuit, and an electroacoustic transducer in electrical communication with the control circuit, wherein the car-wearable device is configured to calculate one or more desired gait parameters, and provide a series of audio cues to a device wearer consistent with the one or more desired gait parameters.

This application is being filed as a PCT International Patent application on Jun. 21, 2022, in the name of Starkey Laboratories, a U.S. national corporation, applicant for the designation of all countries, and Achintya Kumar Bhowmik, a U.S. Citizen, and Bernard Bechara, a U.S. Citizen, and Justin R. Burwinkel, a U.S. Citizen, and Krishna Chaithanya Vastare, a U.S. Citizen, and Gerard N. Weisensel, a U.S. Citizen, inventors for the designation of all countries, and claims priority to U.S. Provisional Patent Application No. 63/212,906 filed Jun. 21, 2021, the contents of which are herein incorporated by reference in its entirety.

### FIELD

Embodiments herein relate to ear-wearable devices and systems that can analyze a device wearer's gait and/or provide gait training.

### BACKGROUND

Gait refers to an individual's pattern of walking. Walking involves balance and the coordination of muscles so that the body is propelled forward in a rhythm, called the stride. An individual's gait may be abnormal or atypical (acutely or chronically) for a wide variety of reasons. Common causes for an abnormal or atypical gait include degenerative diseases, vestibular system disorders, neurological injuries and conditions, musculoskeletal injuries, weakness and/or pain, poorly fitting footwear, and the like. As a specific example, Parkinson's patients can experience gait freeze. Stroke patients may also exhibit an abnormal or atypical gait.

Since an abnormal or atypical gait may reflect serious health conditions, there is substantial value in tracking and/or analyzing an individual's gait. Further, in some scenarios, it can be useful to provide an individual with gait training or therapy, which may reduce the individual's risks of falling and getting injured.

### SUMMARY

Embodiments herein relate to ear-wearable devices and systems that can analyze a device wearer's gait and/or provide gait training. In a first aspect, an ear-wearable device is included having a control circuit, a motion sensor in electrical communication with the control circuit, a microphone in electrical communication with the control circuit, and an electroacoustic transducer in electrical communication with the control circuit, wherein the ear-wearable device is configured to calculate one or more desired gait parameters, and provide a series of audio cues to a device wearer consistent with the one or more desired gait parameters.

In a second aspect, in addition to one or more of the preceding or following aspects, or in the alternative to some aspects, the series of audio cues include a rhythmic sequence.

In a third aspect, in addition to one or more of the preceding or following aspects, or in the alternative to some aspects, the series of audio cues include speech.

In a fourth aspect, in addition to one or more of the preceding or following aspects, or in the alternative to some aspects, the series of audio cues include virtual spatialized audio.

In a fifth aspect, in addition to one or more of the preceding or following aspects, or in the alternative to some aspects, the one or more desired gait parameters includes one or more of a gait tempo, a gait cadence, step impact magnitude, and a left vs. right symmetry value.

In a sixth aspect, in addition to one or more of the preceding or following aspects, or in the alternative to some aspects, the ear-wearable device is configured to record signals from at least one of the motion sensor and the microphone and process the signals to characterize an existing gait of the device wearer.

In a seventh aspect, in addition to one or more of the preceding or following aspects, or in the alternative to some aspects, the ear-wearable device is configured to calculate the one or more desired gait parameters by evaluating signals from the motion sensor and/or the microphone and referencing stored data regarding target gait parameters.

In an eighth aspect, in addition to one or more of the preceding or following aspects, or in the alternative to some aspects, the target gait parameters are input by the device wearer, the device manufacturer, a care provider, or a medical professional.

In a ninth aspect, in addition to one or more of the preceding or following aspects, or in the alternative to some aspects, the ear-wearable device is configured to calculate the one or more desired gait parameters by evaluating signals from the motion sensor and/or the microphone reflecting a current gait of the device wearer during an observation period.

In a tenth aspect, in addition to one or more of the preceding or following aspects, or in the alternative to some aspects, the ear-wearable device is configured to normalize the one or more desired gait parameters based on a detected activity level as reflected in data from the motion sensor.

In an eleventh aspect, in addition to one or more of the preceding or following aspects, or in the alternative to some aspects, the ear-wearable device is configured to initiate or discontinue the series of audio cues based on a detected activity state as reflected in data from the motion sensor and/or the microphone.

In a twelfth aspect, in addition to one or more of the preceding or following aspects, or in the alternative to some aspects, the detected activity state can include the device wearer assuming a standing or upright posture.

In a thirteenth aspect, in addition to one or more of the preceding or following aspects, or in the alternative to some aspects, the detected activity state can include cessation of the device wearer walking.

In a fourteenth aspect, in addition to one or more of the preceding or following aspects, or in the alternative to some aspects, the ear-wearable device is configured to initiate the series of audio cues based on detection of an abnormal or atypical gait.

In a fifteenth aspect, in addition to one or more of the preceding or following aspects, or in the alternative to some aspects, the ear-wearable device is configured to initiate the series of audio cues based on detection of a gait with a step timing variability or statistics crossing a threshold value.

In a sixteenth aspect, in addition to one or more of the preceding or following aspects, or in the alternative to some aspects, the threshold value is set through evaluation of previous events related to the gait of the device wearer.

In a seventeenth aspect, in addition to one or more of the preceding or following aspects, or in the alternative to some aspects, the threshold value is set through evaluation of previous events including one or more of gait freezes, stumbles, falls, cessation of walking, or continued walking with appreciably the same gait metrics or improved.

In an eighteenth aspect, in addition to one or more of the preceding or following aspects, or in the alternative to some aspects, the ear-wearable device is configured to initiate the series of audio cues based on detection of a gait with a left-right symmetry variability or statistics crossing a threshold value.

In a nineteenth aspect, in addition to one or more of the preceding or following aspects, or in the alternative to some aspects, the ear-wearable device is configured to initiate the series of audio cues based on detection of the presence of a device wearer within a particular environment.

In a twentieth aspect, in addition to one or more of the preceding or following aspects, or in the alternative to some aspects, the particular environment can include an outdoor environment.

In a twenty-first aspect, in addition to one or more of the preceding or following aspects, or in the alternative to some aspects, the particular environment can include an indoor environment.

In a twenty-second aspect, in addition to one or more of the preceding or following aspects, or in the alternative to some aspects, the ear-wearable device is configured to interface with an accessory device and send or receive information regarding the one or more desired gait parameters.

In a twenty-third aspect, in addition to one or more of the preceding or following aspects, or in the alternative to some aspects, the ear-wearable device is configured to evaluate data from the motion sensor and/or microphone over a time period to determine a range of gait tempo values for the device wearer.

In a twenty-fourth aspect, in addition to one or more of the preceding or following aspects, or in the alternative to some aspects, the ear-wearable device is configured to set an audio property related to the series of audio cues based on a hearing loss of the device wearer.

In a twenty-fifth aspect, in addition to one or more of the preceding or following aspects, or in the alternative to some aspects, the audio property can include a volume, frequency specific amplification, frequency shifting, frequency compression, frequency transposition, and noise cancelation.

In a twenty-sixth aspect, in addition to one or more of the preceding or following aspects, or in the alternative to some aspects, the ear-wearable device is configured to adjust an audio property related to the series of audio cues based at least in part on an audiogram or other hearing test of the device wearer.

In a twenty-seventh aspect, in addition to one or more of the preceding or following aspects, or in the alternative to some aspects, the ear-wearable device is configured to evaluate a response of the device wearer in response to the series of audio cues.

In a twenty-eighth aspect, in addition to one or more of the preceding or following aspects, or in the alternative to some aspects, the ear-wearable device is configured to evaluate a response of the device wearer in response to the series of audio cues and adjust the series of audio cues accordingly.

In a twenty-ninth aspect, in addition to one or more of the preceding or following aspects, or in the alternative to some aspects, the ear-wearable device is configured to distinguish between a right step and a left step based on an input received from an accessory device.

In a thirtieth aspect, in addition to one or more of the preceding or following aspects, or in the alternative to some aspects, the ear-wearable device is configured to distinguish between a right step and a left step based on a signal from the motion sensor.

In a thirty-first aspect, in addition to one or more of the preceding or following aspects, or in the alternative to some aspects, the ear-wearable device is configured to characterize a gait of the device wearer at varying levels of physical exertion.

In a thirty-second aspect, in addition to one or more of the preceding or following aspects, or in the alternative to some aspects, the ear-wearable device is configured to characterize a gait of the device wearer at varying levels of cognitive exertion.

In a thirty-third aspect, in addition to one or more of the preceding or following aspects, or in the alternative to some aspects, the ear-wearable device is configured to detect whether the device wearer is changing elevation and changing a tempo of the series of audio cues accordingly.

In a thirty-fourth aspect, in addition to one or more of the preceding or following aspects, or in the alternative to some aspects, the changing elevation can include going up or down stairs or walking up or down a hill.

In a thirty-fifth aspect, in addition to one or more of the preceding or following aspects, or in the alternative to some aspects, the ear-wearable device is configured to sense ambient conditions around the device wearer and change a tempo of the series of audio cues accordingly.

In a thirty-sixth aspect, in addition to one or more of the preceding or following aspects, or in the alternative to some aspects, the ear-wearable device is configured to record identifying wireless packets encountered and cross-reference gait against the recorded identifying wireless packets.

In a thirty-seventh aspect, in addition to one or more of the preceding or following aspects, or in the alternative to some aspects, the identifying wireless packets can include BLUETOOTH advertising packets.

In a thirty-eighth aspect, in addition to one or more of the preceding or following aspects, or in the alternative to some aspects, the ear-wearable device is configured to record third party voices encountered and cross-reference gait against the recorded third party voices.

In a thirty-ninth aspect, an ear-wearable device is included having a control circuit, a motion sensor, a microphone, and an electroacoustic transducer, wherein the ear-wearable device is configured to operate in a first mode, wherein the first mode includes evaluating signals from at least one of the motion sensor and the microphone to characterize a gait of a device wearer, and operate in a second mode, wherein the second mode includes providing a series of audio cues to the device wearer.

In a fortieth aspect, in addition to one or more of the preceding or following aspects, or in the alternative to some aspects, the series of audio cues are configured to reflect an idealized gait specific for the device wearer.

In a forty-first aspect, in addition to one or more of the preceding or following aspects, or in the alternative to some aspects, the idealized gait reflects one or more of an idealized gait tempo, gait cadence, step impact magnitude, and left vs. right symmetry value.

In a forty-second aspect, in addition to one or more of the preceding or following aspects, or in the alternative to some aspects, the series of audio cues exhibit a left-right asymmetry that is characteristic for the device wearer.

In a forty-third aspect, in addition to one or more of the preceding or following aspects, or in the alternative to some aspects, the series of audio cues include a rhythmic sequence.

In a forty-fourth aspect, in addition to one or more of the preceding or following aspects, or in the alternative to some aspects, the series of audio cues include speech.

In a forty-fifth aspect, in addition to one or more of the preceding or following aspects, or in the alternative to some aspects, the series of audio cues include virtual spatialized audio.

In a forty-sixth aspect, in addition to one or more of the preceding or following aspects, or in the alternative to some aspects, the ear-wearable device is configured to evaluate signals from at least one of the motion sensor and the microphone to detect whether the device wearer is walking.

In a forty-seventh aspect, in addition to one or more of the preceding or following aspects, or in the alternative to some aspects, the ear-wearable device is configured to initiate or discontinue the series of audio cues based on a detected activity state as reflected in data from the motion sensor and/or the microphone.

In a forty-eighth aspect, in addition to one or more of the preceding or following aspects, or in the alternative to some aspects, the detected activity state can include the device wearer assuming a standing or upright posture.

In a forty-ninth aspect, in addition to one or more of the preceding or following aspects, or in the alternative to some aspects, the detected activity state can include cessation of the device wearer walking.

In a fiftieth aspect, in addition to one or more of the preceding or following aspects, or in the alternative to some aspects, the ear-wearable device is configured to initiate the series of audio cues based on detection of an abnormal or atypical gait.

In a fifty-first aspect, in addition to one or more of the preceding or following aspects, or in the alternative to some aspects, the ear-wearable device is configured to initiate the series of audio cues based on detection of a gait with a step timing variability or statistics crossing a threshold value.

In a fifty-second aspect, in addition to one or more of the preceding or following aspects, or in the alternative to some aspects, the threshold value is set through evaluation of previous events related to the gait of the device wearer.

In a fifty-third aspect, in addition to one or more of the preceding or following aspects, or in the alternative to some aspects, the ear-wearable device is configured to initiate the series of audio cues based on detection of a gait with a left-right symmetry variability or statistics crossing a threshold value.

In a fifty-fourth aspect, in addition to one or more of the preceding or following aspects, or in the alternative to some aspects, the threshold value is set through evaluation of previous events related to the gait of the device wearer.

In a fifty-fifth aspect, in addition to one or more of the preceding or following aspects, or in the alternative to some aspects, the ear-wearable device is configured to initiate the series of audio cues based on detection of the presence of the device wearer within a particular environment.

In a fifty-sixth aspect, in addition to one or more of the preceding or following aspects, or in the alternative to some aspects, the particular environment can include an outdoor environment.

In a fifty-seventh aspect, in addition to one or more of the preceding or following aspects, or in the alternative to some aspects, the particular environment can include an indoor environment.

In a fifty-eighth aspect, in addition to one or more of the preceding or following aspects, or in the alternative to some aspects, the ear-wearable device, when operating in the first mode, is configured to evaluate data from the motion sensor over a time period to determine a range of gait tempo values for the device wearer.

In a fifty-ninth aspect, in addition to one or more of the preceding or following aspects, or in the alternative to some aspects, the ear-wearable device is configured to evaluate a response of the device wearer in response to the series of audio cues.

In a sixtieth aspect, in addition to one or more of the preceding or following aspects, or in the alternative to some aspects, the ear-wearable device is configured to evaluate a response of the device wearer in response to the series of audio cues and adjust the series of audio cues accordingly.

In a sixty-first aspect, in addition to one or more of the preceding or following aspects, or in the alternative to some aspects, the ear-wearable device is configured to distinguish between a right step and a left step based on an input received from an accessory device.

In a sixty-second aspect, in addition to one or more of the preceding or following aspects, or in the alternative to some aspects, the ear-wearable device is configured to match the series of audio cues to the characterized gait of the device wearer.

In a sixty-third aspect, in addition to one or more of the preceding or following aspects, or in the alternative to some aspects, the ear-wearable device is configured to prompt the device wearer to execute specific actions while operating in the first mode. In a sixty-fourth aspect, in addition to one or more of the preceding or following aspects, or in the alternative to some aspects, the specific actions can include a movement protocol.

In a sixty-fifth aspect, in addition to one or more of the preceding or following aspects, or in the alternative to some aspects, the specific actions are performed while the device wearer's eyes are closed.

In a sixty-sixth aspect, in addition to one or more of the preceding or following aspects, or in the alternative to some aspects, the ear-wearable device is configured to set an audio property related to the series of audio cues based on a hearing loss of the device wearer.

In a sixty-seventh aspect, in addition to one or more of the preceding or following aspects, or in the alternative to some aspects, the audio property can include a volume, frequency specific amplification, frequency shifting, frequency compression, frequency transposition, and noise cancelation.

In a sixty-eighth aspect, in addition to one or more of the preceding or following aspects, or in the alternative to some aspects, the ear-wearable device is configured to adjust an audio property related to the series of audio cues based at least in part on an audiogram or other hearing test of the device wearer.

In a sixty-ninth aspect, an ear-wearable device is included having a control circuit, a motion sensor, a microphone, and an electroacoustic transducer, wherein the ear-wearable device is configured to generate a set of data reflecting a current gait of a device wearer based on signals from at least one of the motion sensor and the microphone, and match the set of data against a plurality of predetermined patterns to characterize the current gait.

In a seventieth aspect, in addition to one or more of the preceding or following aspects, or in the alternative to some aspects, the ear-wearable device is configured to match the set of data against a plurality of predetermined patterns to characterize a current health status of the device wearer.

In a seventy-first aspect, in addition to one or more of the preceding or following aspects, or in the alternative to some aspects, the ear-wearable device is configured to determine whether the characterized current gait reflects a musculoskeletal injury or imbalance.

In a seventy-second aspect, in addition to one or more of the preceding or following aspects, or in the alternative to some aspects, the ear-wearable device is configured to alert the device wearer and/or a third party regarding the musculoskeletal injury or imbalance.

In a seventy-third aspect, in addition to one or more of the preceding or following aspects, or in the alternative to some aspects, the ear-wearable device is configured to generate a suggestion regarding a physical activity to ameliorate the musculoskeletal injury or imbalance.

In a seventy-fourth aspect, in addition to one or more of the preceding or following aspects, or in the alternative to some aspects, the ear-wearable device is configured to identify whether the device wearer is using a walking assistance device.

In a seventy-fifth aspect, in addition to one or more of the preceding or following aspects, or in the alternative to some aspects, the walking assistance device can include a cane, a walker, a knee walker, or crutches.

In a seventy-sixth aspect, in addition to one or more of the preceding or following aspects, or in the alternative to some aspects, the ear-wearable device is configured to identify a condition of hypokinetic feet based on the characterization of the current gait.

In a seventy-seventh aspect, in addition to one or more of the preceding or following aspects, or in the alternative to some aspects, the ear-wearable device is configured to characterize a current emotional status of the device wearer based on the current gait of the device wearer.

In a seventy-eighth aspect, an ear-wearable device is included having a control circuit, a motion sensor, a microphone, and an electroacoustic transducer, wherein the ear-wearable device is configured to generate a set of data reflecting a current gait of a device wearer based on signals from at least one of the motion sensor and the microphone, compare the set of data against stored data reflecting a previous gait of the device wearer, and characterize a health status of the device wearer based on a change from the previous gait to the current gait of the device wearer.

In a seventy-ninth aspect, in addition to one or more of the preceding or following aspects, or in the alternative to some aspects, the ear-wearable device is configured to determine whether the change from the previous gait to the current gait reflects an injury.

In an eightieth aspect, in addition to one or more of the preceding or following aspects, or in the alternative to some aspects, the ear-wearable device is configured to determine whether the change from the previous gait to the current gait reflects a neurological disease state or a neurological injury.

In an eighty-first aspect, in addition to one or more of the preceding or following aspects, or in the alternative to some aspects, the ear-wearable device is configured to determine whether the change from the previous gait to the current gait reflects an elevated fall risk.

In an eighty-second aspect, in addition to one or more of the preceding or following aspects, or in the alternative to some aspects, the ear-wearable device is configured to initiate audio cues for delivery to the device wearer when an elevated fall risk is present.

In an eighty-third aspect, in addition to one or more of the preceding or following aspects, or in the alternative to some aspects, the device wearer is configured to send a control signal to a secondary device when an elevated fall risk is present.

In an eighty-fourth aspect, in addition to one or more of the preceding or following aspects, or in the alternative to some aspects, the secondary device can include a home automation device.

In an eighty-fifth aspect, in addition to one or more of the preceding or following aspects, or in the alternative to some aspects, the ear-wearable device is configured to determine whether the change from the previous gait to the current gait reflects a reduced fall risk.

In an eighty-sixth aspect, in addition to one or more of the preceding or following aspects, or in the alternative to some aspects, the ear-wearable device is configured to determine whether the change from the previous gait to the current gait reflects an improved health state.

In an eighty-seventh aspect, in addition to one or more of the preceding or following aspects, or in the alternative to some aspects, the ear-wearable device is configured to send a notification of the health status of the device wearer to a third party.

In an eighty-eighth aspect, in addition to one or more of the preceding or following aspects, or in the alternative to some aspects, the ear-wearable device is configured to determine whether the change from the previous gait to the current gait reflects a slowing gait tempo.

In an eighty-ninth aspect, in addition to one or more of the preceding or following aspects, or in the alternative to some aspects, the ear-wearable device is configured to cross-reference changes in gait with changes in activity levels of the device wearer.

In a ninetieth aspect, in addition to one or more of the preceding or following aspects, or in the alternative to some aspects, the ear-wearable device is configured to cross-reference changes in gait with changes in footwear of the device wearer as identified by at least one of signals from the microphone and input from the device wearer.

In a ninety-first aspect, an ear-wearable system is included having a first ear-wearable device, the first ear-wearable device can include a first control circuit, a first motion sensor, a first microphone, and a first electroacoustic transducer. The ear-wearable system further includes a second ear-wearable device, the second ear-wearable device can include a second control circuit, a second motion sensor, a second microphone, and a second electroacoustic transducer. The ear-wearable system is configured to calculate one or more desired gait parameters and provide a series of audio cues to a device wearer consistent with the one or more desired gait parameters.

In a ninety-second aspect, in addition to one or more of the preceding or following aspects, or in the alternative to some aspects, the series of audio cues are delivered differentially through the first ear-wearable device and the second ear-wearable device.

In a ninety-third aspect, in addition to one or more of the preceding or following aspects, or in the alternative to some aspects, the ear-wearable system is configured to compare signals from the first ear-wearable device and the second ear-wearable device to discriminate between a right side footfall and a left side footfall.

In a ninety-fourth aspect, in addition to one or more of the preceding or following aspects, or in the alternative to some aspects, the ear-wearable system is configured to duty cycle operations between the first ear-wearable device and the second ear-wearable device.

In a ninety-fifth aspect, in addition to one or more of the preceding or following aspects, or in the alternative to some aspects, the series of audio cues can include virtual spatialized audio.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of an ear-wearable device in accordance with various embodiments herein.

FIG. 2 is a schematic view of an ear-wearable device in the ear of a device wearer in accordance with various embodiments herein.

FIG. 3 is a schematic view of device wearer in accordance with various embodiments herein.

FIG. 4 is a schematic view of elements of gait in accordance with various embodiments herein.

FIG. 5 is a schematic top view of a device wearer in accordance with various embodiments herein.

FIG. 6 is a schematic view of left and right-side steps in accordance with various embodiments herein.

FIG. 7 is a schematic view of left and right-side steps in accordance with various embodiments herein.

FIG. 8 is a schematic view of left and right-side steps in accordance with various embodiments herein.

FIG. 9 is a schematic view of a device wearer getting up from a chair in accordance with various embodiments herein.

FIG. 10 is a schematic view of environments in accordance with various embodiments herein.

FIG. 11 is a schematic view of a device wearer illustrating characterization of a current gait to detect a health status, an injury, or another condition.

FIG. 12 is a schematic view of an accessory device in accordance with various embodiments herein.

FIG. 13 is a schematic block diagram illustrating various components of an ear-wearable device in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

There is substantial value in tracking and/or analyzing an individual's gait. Further, in some scenarios, it can be useful to provide an individual with gait training or therapy. For example, a stroke patient may be able to improve their gait over time with consistent gait training or therapy. Further, patients with Parkinson's disease who may experience a gait freeze can benefit from gait training or therapy. Tracking an individual's gait patterns to detect deteriorations and/or providing gait training or therapy may reduce the individual's risks of falling and getting injured.

Embodiments of ear-wearable device herein can be used to provide gait analysis, training and/or therapy. Various embodiments of ear-wearable devices herein can specifically analyze a device wearer's gait and/or provide a series of cues to the device wearer in the context of providing gait training and/or therapy.

Ear-wearable devices herein are uniquely capable of, and valuable for, analyzing a device wearer's gait and/or providing gait training. This is because such devices, including those used as hearing assistance devices, are typically worn all the time (or nearly all the time) by device wearers. This means that analyzing gait can be done in a natural setting and reflect the device wearer's true gait with a higher level of accuracy. In addition, analyzing gait can be performed much more often to allow changes in gait to be more quickly and accurately recognized. Further, gait training/therapy can be initiated at times that are ideal (such as when the wearer is already walking) without requiring the device wearer to recognize such an opportunity in advance and remember to carry/use a specific device capable of providing gait training/therapy. In addition, ear-wearable devices herein, by virtue of being wearable on, about or in the ears, can provide cues, such as audio cues, that are received by the device wearer, but not perceptible by other individuals who may be near the device wearer. As such, ear-wearable devices herein can provide gait training in a discreet manner.

In accordance with various embodiments herein, two spatially-separated ear-wearable devices can be used (e.g., one associated with each ear) offering a number of benefits including duty cycle like splitting of operations for more efficient battery usage as well as the ability to more finely discriminate between left-side and right-side events such as left-side versus right-side footfalls.

Embodiments herein include ear-wearable devices that can calculate one or more desired gait parameters and then provide a series of audio cues to a device wearer consistent with the one or more desired or target gait parameters.

Embodiments herein can also include ear-wearable devices configured to operate in a first mode and second mode, wherein the first mode includes evaluating signals from sensors, such as at least one of a motion sensor and a microphone, to characterize a current gait of a device wearer and the second mode includes providing a series of audio cues to the device wearer to influence and/or change the gait of the device wearer.

Embodiments herein can also include ear-wearable devices configured to generate a set of data reflecting a current gait of a device wearer based on signals from at least one sensor, such as at least one of the motion sensor and the microphone, and match the set of data against a plurality of predetermined patterns to characterize the current gait.

Embodiments herein can also include ear-wearable devices configured to generate a set of data reflecting a current gait of a device wearer based on signals from at least one sensor, such as at least one of the motion sensor and the microphone, compare the set of data against stored data reflecting a previous gait of the device wearer, and characterize a health status of the device wearer based on a change from the previous gait to the current gait of the device wearer.

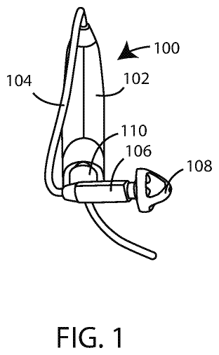

Referring now to FIG. 1, a schematic view of an ear-wearable device 100 is shown in accordance with various embodiments herein. The ear-wearable device 100 includes a housing 102 in which various components of the device can be housed. In this example, the ear-wearable device 100 also includes a battery compartment 110. However, some types of ear-wearable devices herein may lack a battery compartment, such as a device with a rechargeable battery. The ear-wearable device 100 also includes a cable 104 which connects to a receiver 106. The ear-wearable device 100 also includes an earbud 108. In various embodiments, the ear-wearable device 100 can provide cues to the device wearer to improve, maintain, or otherwise change their gait as well as elements contributing to their gait.

The cues provided by the device can take on many different forms. In some embodiments, the cues can be audio cues, haptic cues, and/or (such as in combination with a separate device) visual cues. In various embodiments, the series of cues can include a rhythmic sequence. In some embodiments, the series of cues can include a metronome-like series of beats. In some embodiments, the cues can include music with a beat serving as a series of cues. In various embodiments, the series of cues can take the form of an audio cue that is similar to the sound of a percussion instrument or another musical instrument. In various embodiments, the series of cues can take the form of audible speech cues such as “left”, “right”, “left”, “right”, etc. or other spoken words.

In some embodiments, the series of cues can take the form of virtual spatialized sounds (e.g., sounds delivered in a manner to provide the perception of having a specific spatial origin), details of which are provided in greater detail below.

In some embodiments, the cues can be provided as a series of separate sounds interrupted by periods of silence. For example, a cue can be provided as a sound lasting for a particular duration. In some embodiments, the duration can be greater than or equal to 100 milliseconds, 300 milliseconds, 500 milliseconds, 700 milliseconds, 900 milliseconds, 1100 milliseconds, 1300 milliseconds, 1500 milliseconds, 1700 milliseconds, 1900 milliseconds, 2100 milliseconds, 2300 milliseconds, 2500 milliseconds, 2700 milliseconds, 2900 milliseconds, or 3000 milliseconds, or can be an amount falling within a range between any of the foregoing. However, in some embodiments, the cues can be provided as overlayed on a continuous sound stream, such as a beat provided with music.

In some embodiments, the cues provided by the ear-wearable device 100 can serve to assist the device wearer in exhibiting a gait that matches or is closer to desired or target gait parameters. Thus, in various embodiments, the ear-wearable device 100 can be configured to provide a series of audio and/or haptic cues to a device wearer consistent with the one or more desired or target gait parameters. In various embodiments, the one or more desired gait or target parameters includes one or more of a desired or target gait tempo, a gait cadence, step impact magnitude, a left vs. right symmetry value, stride height, or the like.

In some embodiments, the ear-wearable device 100 can calculate or otherwise determine desired or target gait parameters. For example, in some embodiments, the ear-wearable device 100 can be configured to calculate one or more desired or target gait parameters by evaluating signals from a motion sensor and referencing stored data regarding target gait parameters. In various embodiments, the ear-wearable device 100 can be configured to calculate one or more desired gait parameters by evaluating signals from a motion sensor reflecting a current gait of a device wearer during an observation period. In some embodiments, the desired or target gait parameter may reflect a perfectly symmetrical and consistent gait. However, recognizing that a perfect gait may not be realistic of a large number of device wearers, in some embodiments, the desired or target gait parameter may reflect an improvement (e.g., greater left right symmetry, more consistent gait tempo or cadence, or the like) over the current gait with respect to one or more gait parameters discussed herein.

In some embodiments, the desired or target gait parameters are input into ear-wearable device 100 by an individual such as the device wearer, a care provider, the device manufacturer, or a medical professional.

Gait training therapy herein may only be provided for a portion of the time that the ear-wearable device is in use by the device wearer. For example, in the context of an ear-wearable device that serves as a hearing assistance device, gait training may be a feature that is only selectively initiated and thus only provided some of the time that the device may be worn.

Gait training therapy (such as providing a series of gait cues) herein can be initiated in various ways. In some embodiments, gait training can be initiated based on a command or input from an individual such as a device wearer, a care provider, a clinician, or the like. In some embodiments, gait training can be initiated based on a predetermined schedule. For example, gait training can be initiated at certain time every day. In some embodiments, gait training can be initiated when certain conditions (with respect to the device wearer and/or the environment of the device wearer) are detected.

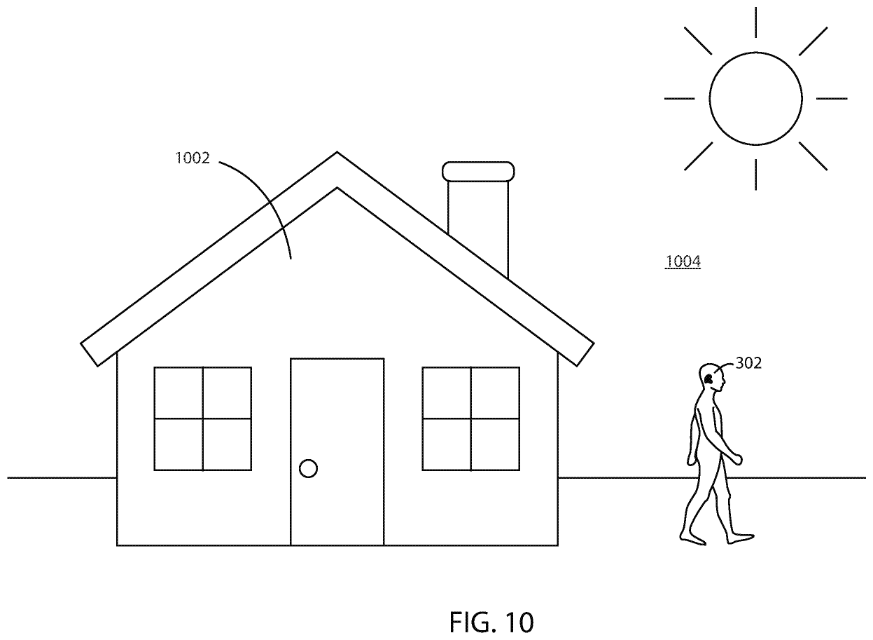

In some embodiments, gait training can be initiated by the ear-wearable device or system itself dynamically. For example, in various embodiments, the ear-wearable device 100 can be configured to initiate or discontinue a series of audio cues based on a detected activity state as reflected in data from a motion sensor and/or a microphone. In various embodiments, the ear-wearable device 100 can be configured to initiate a series of audio cues based on detection of an abnormal or atypical gait. In various embodiments, the ear-wearable device 100 can be configured to initiate a series of audio cues based on detection of a gait with a step timing variability or statistics crossing a threshold value. In various embodiments, the ear-wearable device 100 can be configured to initiate a series of audio cues based on detection of a gait with a left-right symmetry variability or statistics (in one or more gait parameters) crossing a threshold value. In various embodiments, the ear-wearable device 100 can be configured to initiate a series of audio cues based on detection of the presence of a device wearer within a particular environment, such as described with respect to FIG. 10 below.

In various embodiments herein, the device or system can provide a notice to the device wearer that gait training has been or will be initiated. For example, using a receiver and/or electroacoustic transducer of the ear-wearable device an audible notice can be provided such as “gait training will begin in 10 seconds”. In various embodiments, the device or system can monitor for user feedback/input regarding whether or not they want gait training to begin. For example, the device can receive input from the device wearer, either through the microphone, a device “tap”, a user input received through an accessory device, or the like to cancel, delay or reschedule the gait training.

Many different adjustments to the series of cues can be made. By way of example, in some embodiments, the ear-wearable device 100 can be configured to adjust a volume or other aspects related to a series of audio cues. In some embodiments, the ear-wearable device 100 can be configured to set or adjust an audio property related to the series of audio cues based on a hearing loss of the device wearer. In this manner, individuals with a hearing loss can be provided with audio cues in a manner they can hear and understand. In some embodiments, the audio property set or adjusted can include a volume, frequency specific amplification, frequency shifting, frequency compression, frequency transposition, and noise cancelation. In some embodiments, the ear-wearable device can specifically adjust for frequency-specific volume selections, differential signal generation considering non-linear loudness perceptions (recruitment) measured or inferred for the particular user, avoidance of providing audio in inaudible ranges/cochlear dead-regions that are measured or inferred for the particular user, and/or provide enough spectral separation between similar frequency tones such that the individual is able to distinguish between them based on either measurements or inferences. In some embodiments the ear-wearable device 100 is configured to adjust an audio property related to the series of audio cues based at least in part on an audiogram or other hearing test of the device wearer. In some embodiments, an audiogram or other results of hearing evaluation can be provided through interface with a database or a system such as an electronic medical records system. In some embodiments, an audiogram or other results of hearing evaluation can be obtained through user input (such as third-party user input). In some embodiments, an audiogram and/or data approximating an audiogram and/or other results of hearing evaluation can be generated using a testing procedure implemented with the device itself. For example, the ear-wearable device 100 can play sounds or tones and receive feedback from the device wearer regarding what is heard.

In some embodiments, the ear-wearable device 100 can be configured to adjust a tempo of a series of audio cues. In some embodiments, the ear-wearable device 100 can be configured to adjust cues for a right side and a left side differently. For example, in some embodiments, the ear-wearable device 100 can adjust audio cues for one side (right or left) to be at a different volume, a different pitch, a different duration, or the like versus the other side.

In various embodiments, the ear-wearable device 100 can be configured to evaluate a response of a device wearer in response to a series of audio cues. In various embodiments, the ear-wearable device 100 can be configured to evaluate a response of a device wearer in response to a series of audio cues and adjust the series of audio cues accordingly. By way of example, the ear-wearable device 100 can analyze the device wearer's gait as influenced by the cues provided. If the gait has improved (such as being more symmetrical, more consistent, faster, etc.), then in some embodiments the device can provide cues that represent an additional challenge such as being closer to a gait with perfect symmetry, being faster in tempo, etc. However, if the gait has not improved or has gotten worse, then the device can continue to provide cues in the same manner as before or provide cues that are less challenging in terms of their symmetry, tempo, etc.

It will be appreciated that every individual is different and may have physical and/or neurological characteristics that impact their gait. Some of these characteristics may prevent an individual from achieving a gait, even with training, that is a perfectly symmetrical gait. Therefore, in some embodiments, the series of cues can be configured to reflect an idealized gait that is specific for the device wearer. In various embodiments, the ear-wearable device 100 wherein a series of audio cues exhibit a left-right asymmetry that is characteristic for a device wearer. In various embodiments, the ear-wearable device 100 can be configured to match a series of audio cues to the characterized gait of a device wearer.

Regardless of a starting point or baseline gait for a device wearer, changes in gait can be very important in evaluating the condition of a device wearer. Such changes can reflect improvements reflecting a positive prognosis but can also reflect declines reflecting a negative prognosis and/or a health status requiring urgent intervention. It can be clinically valuable to identify changes that occur over a relatively long period of time (e.g., over days, weeks, months, etc.) however it may be even more critical to identify rapid changes (e.g., changes occurring over a period of seconds or minutes) that may suggest a health condition change requiring urgent intervention.

In various embodiments, the ear-wearable device 100 can be configured to generate a set of data reflecting a current gait of a device wearer based on signals from at least one sensor, such as at least one of a motion sensor and a microphone. In various embodiments, the ear-wearable device 100 can be configured to compare a set of data (or statistics thereof) reflecting a current gait of a device wearer against stored data (or statistics thereof) reflecting a previous gait of the device wearer. The stored data reflecting a previous gait (and statistics thereof) may reflect a gait from seconds, minutes, hours, days, weeks or months in the past. In various embodiments, the ear-wearable device 100 can be configured to characterize a health status of a device wearer based on a change from the previous gait to the current gait of the device wearer. Changes in gait can be reflected in any of the parameters (and their related statistics) herein reflecting gait. In some embodiments, changes herein can include those of statistical significance. For example, in some embodiments, changes herein can include those reflecting a p-value of 5% or lower. In some embodiments, changes herein can include those with a 1, 2, 3, or more standard deviation difference from a previous observed value. In various embodiments, the ear-wearable device 100 can be configured to determine whether the change from the previous gait to the current gait reflects a changing (slowing or increasing) gait tempo. In various embodiments, the ear-wearable device 100 can be configured to determine whether the change from the previous gait to the current gait reflects a change in left/right asymmetry. In various embodiments, the ear-wearable device 100 can be configured to determine whether the change from the previous gait to the current gait reflects a change in stride lengths.

In various embodiments, the ear-wearable device 100 can be configured to determine whether the change from the previous gait to the current gait reflects a health status change. In various embodiments, the ear-wearable device 100 can be configured to determine whether the change from the previous gait to the current gait reflects an elevated fall risk. An elevated fall risk may be indicated by one or more of an increase in gait asymmetry, an increase in gait timing variability, reduced gait speed, decreased stride height and length, increased front-back and side-to-side sway, increased double stance time, increased hesitancy, slowed postural transitions, wide or en bloc turns, a wide base, and out-of-plane motion, or other gait characteristics or statistics. In various embodiments, the ear-wearable device 100 can be configured to initiate audio cues for delivery to a device wearer when an elevated fall risk is present. In some embodiments, the ear-wearable device 100 can be configured to cease audio cues when an elevated fall risk is present and/or instruct the device wearer to pause, sit down, or use an assistive device to prevent a possibly injurious fall. In various embodiments, the ear-wearable device 100 can be configured to send an alert to a third party when an elevated fall risk is present. In various embodiments, the ear-wearable device 100 can be configured to send a control signal to a secondary device when an elevated fall risk is present.

In various embodiments, the ear-wearable device 100 can be configured to cross-reference changes in gait with changes in data gathered by other sensors, such as change in activity levels of a device wearer. Activity levels can be detected based on data from various sensors including, but not limited to, motion sensor data. In some embodiments, the ear-wearable device 100 can be configured to normalize one or more desired gait parameters based on a detected activity level as reflected in data from at least one sensor, such as a motion sensor.

In various embodiments, the ear-wearable device 100 can be configured to cross-reference changes in gait with changes in footwear of a device wearer as identified by at least one of signals from a microphone and/or input from the device wearer. For example, different types of footwear can create characteristic sounds associated with foot contact with the ground. Changes in such sounds as picked up by a microphone herein can be interpreted as a change in footwear. In some embodiments, the device can query the device wearer regarding their footwear. In some embodiments, the device or system can detect the use of footwear that may adversely affect gait, such as high heels. In some embodiments, the device and or system can issue a notice or alert if a pattern of a worsening gait coinciding with detection of particular footwear is detected.

Various other aspects related to gait can also be determined based on evaluation of data from sensors of devices or systems herein. For example, in various embodiments, the ear-wearable device 100 can be configured to identify whether a device wearer is using a walking assistance device, such as a cane, crutches, walker, knee walker, etc. As one example of how walking assistance device use can be identified, use of a cane generates a distinctive sound signal associated with the cane rhythmically striking the ground. Similarly, other types of assistive devices could be classified using acoustic and motion patterns. In some embodiments, machine learning techniques may be used to train the system on the sound/motion of the individual using their assistive device versus not using their assistive device. In some embodiments, such training can be performed as part of a calibration event. In some embodiments, the sound/motion of the individual using particular assistive devices can be confirmed by way of a query to the user thereby labeling the data and facilitating the use of a supervised machine learning approach. This sound signal can be picked up using a microphone or another sensor herein. For some individuals, they may be more likely to use a walking assistance device when they feel unsteady. As such, the total amount of walking assistance device use can be tracked by the device or system over a period of time and data and/or trends regarding the same can be calculated and/or reported by way of an alert or other communication to a third party such as a care provider and/or a clinician. In some embodiments, if the device and/or system detects that the device wearer regularly uses a cane or other walking assistance device and then later detects the device wearer is not presently using such a device, then the ear-wearable device or system herein can provide a reminder (such as an audible notification) to use the cane or other walking assistance device. In some embodiments, near-falls or stumbles can be detected through the evaluation of sensor data including, but not limited to, motion sensor and/or microphone data over a period of time and data and/or trends regarding the same can be calculated and/or reported by way of an alert or other communication to a third party such as a care provider and/or a clinician.

It will be appreciated that while FIG. 1 illustrates one type of ear-wearable device consistent with embodiments herein, many other types of ear-wearable devices are also contemplated. The term “ear-wearable device” as used herein shall refer to devices that can aid a person with impaired hearing. The term “ear-wearable device” shall also refer to devices that can produce optimized or processed sound for persons with normal hearing. Ear-wearable devices herein can include hearing assistance devices. Ear-wearable devices herein can include, but are not limited to, behind-the-ear (BTE), in-the ear (ITE), in-the-canal (ITC), invisible-in-canal (IIC), receiver-in-canal (RIC), receiver in-the-ear (RITE) and completely-in-the-canal (CIC) type hearing assistance devices. In some embodiments, the ear-wearable device can be a hearing aid falling under 21 C.F.R. § 801.420. In another example, the ear-wearable device can include one or more Personal Sound Amplification Products (PSAPs). In another example, the ear-wearable device can include one or more cochlear implants, cochlear implant magnets, cochlear implant transducers, and cochlear implant processors. In another example, the hearing assistance device can include one or more “hearable” devices that provide various types of functionality. In other examples, ear-wearable devices can include other types of devices that are wearable in, on, or in the vicinity of the user's ears. In other examples, ear-wearable devices can include other types of devices that are implanted or otherwise osseointegrated with the user's skull; wherein the device is able to facilitate stimulation of the wearer's ears via a bone conduction pathway. In another example, the hearing assistance device can include an auditory brainstem implant, a cranial nerve (e.g., CN VIII) implant, and the like.

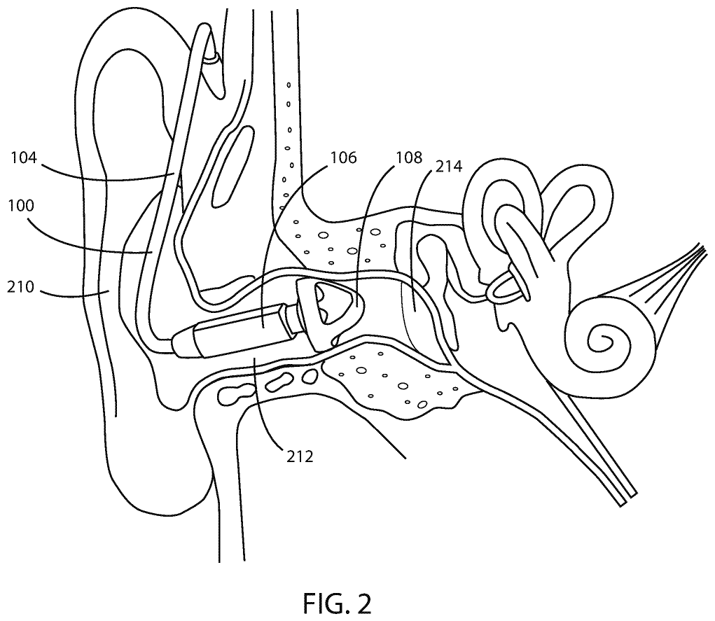

Referring now to FIG. 2, a schematic view of the ear-wearable device 100 is shown with the device fitted in the ear of a device wearer. The significant portions of the ear, in this view, include pinna 210, ear canal 212, and tympanic membrane 214. As before, the ear-wearable device 100 includes a cable 104 connecting to a receiver 106. The ear-wearable device 100 also includes an earbud 108.

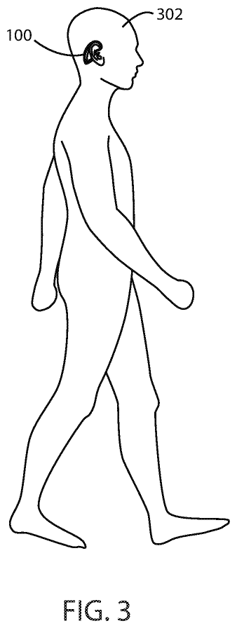

Referring now to FIG. 3, a schematic view of a device wearer 302 with an ear-wearable device 100 is shown in accordance with various embodiments herein. In various embodiments, the ear-wearable device 100 can evaluate signals from at least one sensor (such as a motion sensor and/or a microphone) to detect activity of the device wearer. In various embodiments, the ear-wearable device 100 can evaluate signals from at least one sensor to detect whether the device wearer is specifically walking, such as illustrated in FIG. 3.

In many embodiments herein, it can be valuable to characterize the existing gait of the device wearer. In some embodiments, the ear-wearable device 100 can initiate characterizing the existing gait of the device wearer if the device detects that the device wearer is walking. However, it will be appreciated that the ear-wearable device 100 can also initiate characterizing the existing gait of the device wearer in other scenarios.

Characterizing the existing gait of the device wearer can be done in various ways. In some embodiments, the ear-wearable device 100 can be configured to record signals from a sensor, such as at least one of a motion sensor and a microphone, and then process the signals to characterize an existing gait of a device wearer.

In some scenarios, a device wearer's gait may appear normal/typical at a low level of exertion but may become more abnormal/atypical as the device wearer reaches higher levels of exertion (physical and/or cognitive).

In various embodiments herein, the ear-wearable device 100 can be configured to characterize a gait of a device wearer at varying levels of physical exertion. By way of example, physical exertion can be correlated with heart rate. Heart rate can be determined by a heart rate sensor as described herein. In some embodiments, a characterization of a device wearer's gait can be specific for a given level of physical exertion or heart rate. In various embodiments, the ear-wearable device 100 can characterize gait and/or store gait data as indexed based on exertion level. For example, the device and/or system can characterize gait and/or store gait data as indexed based on heart rate as a proxy for exertion. As a specific example, the device and/or system can characterize gait in a first exertion category reflecting a heart rate of less than about 120 beats per minute as well as in a second exertion category reflecting a heart rate of greater than or equal to 120 beats per minute (or another specific threshold value such as 50, 60, 70, 80, 90, 100, 110, 120, 130, 140, 150, 160, 170, or 180 beats per minute). In some embodiments, the number of categories for exertion indexed categories can be 2, 3, 4, 5, 6, 7, 8, or more using any of the foregoing threshold values to delineate categories or other numbers falling therebetween. Exertion can also be calculated based on motion sensor and/or microphone based activity monitoring and classifications. In some embodiments, gait can be characterized and/or stored while being normalized for exertion on a continuous scale. Similarly, in various embodiments, the ear-wearable device 100 can be configured to characterize a gait of a device wearer at varying levels of cognitive exertion. Cognitive exertion can be associated with certain environments (such as work, school, or the like). In some embodiments, the ear-wearable device 100 can characterize gait and/or store gait data and/or statistics as a function of (or indexed by) location or environment. Cognitive exertion can also be associated with performing multiple tasks simultaneously, such as walking and talking. As such, in some embodiments, other detected device wearer activity (such as talking) can be used as a proxy for cognitive exertion.

In various embodiments, the device can be configured to detect the device wearer's own voice (as opposed to a third party voice) in order to provide a better proxy for cognitive exertion of the device wearer. Own voice detection can be performed in various ways. In some embodiments, this can be performed through signal analysis of the signals generated from the microphone(s). For example, in some embodiments, this can be done by filtering out frequencies of sound that are not associated with speech of the device-wearer. In some embodiments, such as where there are two or more microphones (on the same ear-wearable device or on different ear-wearable devices) this can be done through spatial localization of the origin of the speech or other sounds and filtering out, spectrally subtracting, or otherwise discarding sounds that do not have an origin within the device wearer. In some embodiments, such as where there are two or more ear-worn devices, own-voice detection can be performed and/or enhanced through correlation or matching of intensity levels and or timing.

In some cases, the system can include a bone conduction microphone to preferentially pick up the voice of the device wearer. In some cases, the system can include a directional microphone that is configured to preferentially pick up the voice of the device wearer. In some cases, the system can include an intracanal microphone (a microphone configured to be disposed within the ear-canal of the device wearer) to preferentially pick up the voice of the device wearer. In some cases, the system can include a motion sensor (e.g., an accelerometer configured to be on or about the head of the wearer) to preferentially pick up skull vibrations associated with the vocal productions of the device wearer.

In some cases, an adaptive filtering approach can be used. By way of example, a desired signal for an adaptive filter can be taken from a first microphone and the input signal to the adaptive filter is taken from the second microphone. If the hearing aid wearer is talking, the adaptive filter models the relative transfer function between the microphones. Own-voice detection can be performed by comparing the power of an error signal produced by the adaptive filter to the power of the signal from the standard microphone and/or looking at the peak strength in the impulse response of the filter. The amplitude of the impulse response should be in a certain range to be valid for the own voice. If the user's own voice is present, the power of the error signal will be much less than the power of the signal from the standard microphone, and the impulse response has a strong peak with an amplitude above a threshold. In the presence of the user's own voice, the largest coefficient of the adaptive filter is expected to be within a particular range. Sound from other noise sources results in a smaller difference between the power of the error signal and the power of the signal from the standard microphone, and a small impulse response of the filter with no distinctive peak. Further aspects of this approach are described in U.S. Pat. No. 9,219,964, the content of which is herein incorporated by reference.

In another approach, the system uses a set of signals from a number of microphones. For example, a first microphone can produce a first output signal A from a filter and a second microphone can produce a second output signal B from a filter. The apparatus includes a first directional filter adapted to receive the first output signal A and produce a first directional output signal. A digital signal processor is adapted to receive signals representative of the sounds from the user's mouth from at least one or more of the first and second microphones and to detect at least an average fundamental frequency of voice (pitch output) F0. A voice detection circuit is adapted to receive the second output signal B and the pitch output F0 and to produce an own voice detection trigger T. The apparatus further includes a mismatch filter adapted to receive and process the second output signal B, the own voice detection trigger T, and an error signal E, where the error signal E is a difference between the first output signal A and an output O of the mismatch filter. A second directional filter is adapted to receive the matched output O and produce a second directional output signal. A first summing circuit is adapted to receive the first directional output signal and the second directional output signal and to provide a summed directional output signal (D). In use, at least the first microphone and the second microphone are in relatively constant spatial position with respect to the user's mouth, according to various embodiments. Further aspects of this approach are described in U.S. Pat. No. 9,210,518, the content of which is herein incorporated by reference.

In some embodiments, cognitive exertion can be determined by reference to reaction speed as a proxy, wherein greater cognitive exertion corresponds with slower reaction speeds. Reaction speed can be measured in various ways including providing a stimulus (audio, visual, and/or haptic) and measuring the amount of time for the device wearer to respond to the same. Further details of measuring reaction speed are described in PCT Publ. Appl. No. WO2021/016094, the content of which is herein incorporated by reference.

Some elements of gait irregularity may become more pronounced if the device wearer closes their eyes, such that visual stimuli are not used to maintain their balance. For example, if gait and/or balance becomes significantly worse with their eyes closed, this may indicate that a vestibular system problem is impacting their gait. In various embodiments herein, the ear-wearable device 100 can issue a direction to the device wearer to temporarily close their eyes while specific actions are performed. By way of example, in some embodiments, the ear-wearable device 100 can instruct the device wearer to close their eyes and execute a movement protocol or stand still or close their eyes temporarily while they are walking and compare gait parameters as measured with their eyes open versus as measured with their eyes closed.

In some embodiments, the ear-wearable device 100 may initiate gait training can be initiated based on detection of a specific activity. For example, in many cases, it may be most convenient for the device wearer if gait training is initiated while the device wearer is already walking. Thus, in some embodiments herein, the ear-wearable device 100 can initiate gait training after the device detects that the device wearer is walking. Walking can be detected in various ways including based on observing a rhythmic pattern in sensor signals (such as motion sensor signals) consistent with a step frequency that falls within a normal range for walking or using a pattern matching technique described in greater detail below. In various embodiments, the ear-wearable device 100 can initiate gait training after the device detects that the device wearer is walking for an amount of time crossing a threshold value. For example, the ear-wearable device 100 can initiate gait training after the device detects that the device wearer is walking for a period of time greater than or equal to 0.5, 1, 2, 3, 4, 5, 6, 7, 8, 9, or 10 minutes or more, or an amount falling within a range between any of the foregoing.

In some embodiments, threshold values and/or the data compared with threshold values herein can be ‘log-transformed’ to help account for varied amounts of device use time and other factors. Log-transformed values can be calculated by taking the logarithm of particular values.

In some embodiments herein, the device or system learns a wearer's gait traits and develops a cue scheme (e.g., tempo, cadence) based on the user's gait traits. The system may, for example, learn a set of desirables parameter settings or ranges for a particular user and use those parameter settings or ranges to deliver auditory cues to the wearer via a set of ear-wearable devices. The parameters may include, for example, a tempo (e.g., the step speed), a cadence or rhythm, which may be consistent for left and right steps or may vary (e.g., to accommodate a slower left or right step for a particular user, a volume (loudness) of the cue, and the like. In some examples, the gait may be learned during an initiation or learning phase. In other examples, the gait may be learned as the wearer receives auditory cues.

In various embodiments, the ear-wearable device 100 can be configured to operate in a first mode, wherein the first mode includes evaluating signals from at least one sensor (such as a motion sensor or a microphone or any of the other sensors described herein) to characterize a gait of a device wearer. For example, in some embodiments, the ear-wearable device 100 can be configured to evaluate data from the motion sensor over a time period to determine a range of gait tempo values for the device wearer.

In various embodiments, the ear-wearable device 100, when operating in the first mode, is configured to evaluate data from the motion sensor over a time period to determine a range of gait values, such as gait tempo values, for the device wearer. The time period can be greater than or equal to 0.5, 1, 2, 4, 6, 8, 10, 12, 15, 20, 25, 30, 45, or 60 minutes or more, or an amount falling within a range between any of the foregoing. In various embodiments, the ear-wearable device 100 can be configured to prompt a device wearer to execute specific actions while operating in the first mode.

In various embodiments, the ear-wearable device 100 can be configured to operate in a second mode, wherein the second mode includes providing a series of audio cues to a device wearer as described elsewhere herein.

In some embodiments, devices and/or systems herein can have various pediatric applications. By way of example, the gait of a child can change over time as they gain more coordination and strength. In various embodiments herein, the gait of a device wearer herein can be characterized in order to track against developmental milestones. In some embodiments herein, cues can be provided in order to help a device wearer learn to walk.

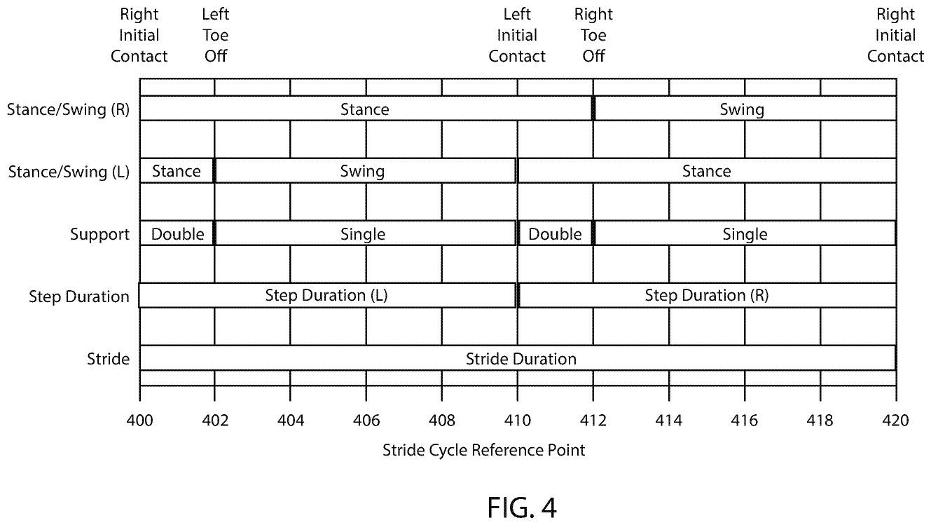

It will be appreciated that a device wearer's stride can be broken down into many different sub-elements for purposes of gait analysis herein. Referring now to FIG. 4, a diagram is shown of events occurring during strides of a device wearer for gait analysis in accordance with various embodiments herein.

At reference point 400, the right foot makes initial contact with the ground (foot fall) and both the right leg and the left leg are in a stance. Support is provided by both legs (i.e., double stance) beginning at this time. At reference point 402, the left toe leaves the ground and the left leg enters a swing while the right leg is in a stance. Support is provided by only the right leg (e.g., single) beginning at this time. At reference points 404, 406, and 408 the swing of the left leg continues. At reference point 410, the left foot makes initial contact with the ground and both the right leg and the left leg are in a stance. Support is provided by both legs beginning at this time. At reference point 412, the right toe leaves the ground and the right leg enters a swing while the left leg is in a stance. Support is provided by only the left leg (e.g., single) beginning at this time. At reference points 414, 416, and 418 the swing of the right leg continues. Reference point 420 marks the conclusion of the stride cycle whereupon if the device wearer continues to walk the cycle will repeat beginning at reference point 400.

In accordance with embodiments herein, one or more of a motion sensor and a microphone herein can detect movements and/or vibrations in order to identify what stage of the stride cycle the device wearer is currently in along with frequencies and time associated with the same. By way of example, reference points 400 and 410 involve the right and left feet, respectively, making initial contact with the ground. The biomechanics associated with such feet/ground contact results in characteristic acoustic and inertial changes that can be detected by one or more microphones and/or accelerometers (or other component) of a motion sensor, either alone or in combination. In some embodiments, characteristics of feet/ground contact can include a signal intensity. In some embodiments, characteristics of feet/ground contact can include a time interval. In some embodiments the spectral intensity and timing of a first, second, third, etc. microphone may be compared, summed, or subtracted to determine the spatial location of a foot fall. In some embodiments, the system may determine if the footfalls are associated with the wearer or if the footfalls are associated with another individual. In some embodiments, the system may also determine if it is the left foot or the right foot making the footfall. In some embodiments, characteristics of feet/ground contact can include an angular position of one or more parts of the body. For example, as one leg swings forward (e.g., starting at reference point 402 and ending at reference point 410 for the left leg and starting at reference point 412 and ending at reference point 420 for the right leg) support by the other leg involves a characteristic vertical motion at a relatively low frequency that can be detected by a component of the motion sensor.

In some embodiments herein, a heart rate or PPG sensor can detect and/or confirm detection of footfalls. A footfall can generate a detectable signal using a heart rate or PPG sensor. In some embodiments, magnitude of motion sensor signals along with heart rate values can be used to differentiate between shuffling, typical walking, and movement due to an external force. This is because the signal from a heart rate or PPG sensor will vary depending on whether the device wearer is shuffling, exhibiting typical walking, or undergoing other movement. In some embodiments, the detection of footfalls with a motion sensor and also with a heart rate or PPG sensor can provide confirmation that the device wearer is actually wearing the ear-wearable devices as intended and not just storing them in their pockets. This is because the devices may still register footfalls with a motion sensor even if the ear-wearable device are not being worn, but a heart rate or PPG sensor associated with the device would not provide a useful signal if the device is not being worn.

Characteristic medio-lateral axis movement can also be detected by the motion sensor during different phases of the stride cycle allowing each point to be identified along with timing of the same. Left versus right steps can also be distinguished by evaluating detected medio-lateral axis movement. By way of example, a limping gait can be reflected as unequal swing durations between each leg and this type of abnormal or atypical gait can be detected by the system. As another example, a shuffling-type gait can be reflected as a measurable variability in the timing of the different phases of the stride cycle that crosses a threshold value of variability or statistics (the threshold value either being pre-selected and programmed into the device or reflecting a statistical measure of deviation from another statistical measure, e.g., an average, for the specific individual as calculated over a look-back period or during a previous calibration period or event). A shuffling-type gait or other scenarios can also be detected using acoustic information obtained from one or more microphones.

In addition, by combining the information content provided by signals associated with directional movement in the horizontal plane (as can be measured by the motion sensor, microphone, or geolocation-type sensors) with that provided by stride cycle analysis as detailed above, aspects such as step length (right, left) and stride length can be calculated. These values can also be subjected to analysis to determine various statistics, e.g., absolute values (average right step length, average left step length, average stride length) as well as ratios of the same (ratio of average right step length vs. average left step length) and measures of variability or other statistics in the same, and the like.

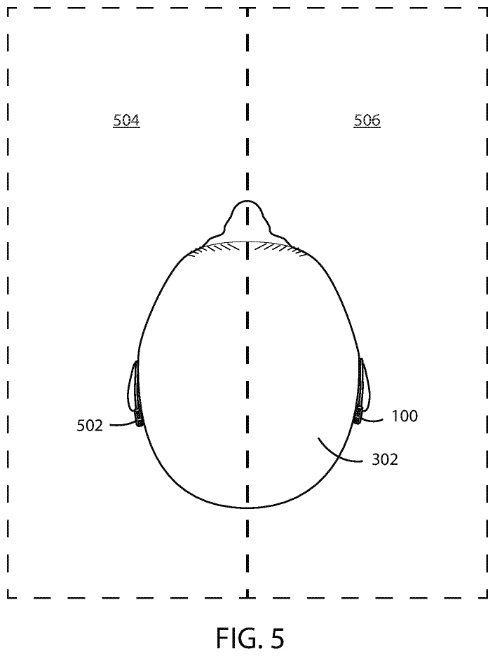

Referring now to FIG. 5, a schematic top view of a device wearer 302 is shown in accordance with various embodiments herein. FIG. 5 shows that the device wearer 302 is wearing an ear-wearable device 100. FIG. 5 illustrates the left side 504 and the right side 506 of the device wearer. The ear-wearable system of this example includes a second ear-wearable device 502.

As such, in various embodiments herein, an ear-wearable system can include a first ear-wearable device 100, including a first control circuit, a first motion sensor, a first microphone, and a first electroacoustic transducer, as well as a second ear-wearable device 502 including a second control circuit, a second motion sensor, a second microphone, and a second electroacoustic transducer. The ear-wearable system can be configured to calculate one or more desired gait parameters and provide a series of audio cues to a device wearer 302 consistent with the one or more desired gait parameters.

In some embodiments, devices and/or systems herein can be configured to determine when the wearer is stepping with a particular foot (i.e., left or right) to enable, e.g., a gait cadence that is tuned for the wearer's gait. For example, if a wearer has a “slow left foot” a device may use a motion sensor, audio information, or both, optionally with other information, to determine when a left step is occurring.

In various embodiments, the ear-wearable device 100 can be configured to distinguish between a right step and a left step based on data from one or more sensors herein. For example, a left step can generally be detected by observing that previous movement toward the left (as part of side-to-side motion during walking) ceases coinciding with motion sensor data associated with the impact of the foot fall and/or microphone data associated with the impact of the footfall. Similarly, a right step can generally be detected by observing that previous movement toward the right ceases coinciding with motion sensor data associated with the impact of the foot fall and/or microphone data associated with the impact of the footfall. In various embodiments, the ear-wearable device 100 can be configured to distinguish between a right step and a left step based on an input received from an accessory device. By way of example, the ear-wearable device 100 can receive a data input from another device, such as a wrist-wearable accessory device or a smart phone to distinguish between a right step and a left step. For example, if the wearer exhibits a typical walking pattern (moving left arm synced with right leg, and vice-versa), the system may receive input or determine whether a wearable device is worn on a left or right arm and use that information to determine when a left (or right) step is occurring. As another example, the device can be configured, in combination with a second device, to distinguish between right and left steps through binaural processing of motion sensor data. The motion sensor of a right ear-wearable device will provide a signal with a slightly different signature upon a right step than the motion sensor of a left ear-wearable device. Thus, the devices and/or system herein can distinguish between a right step and left step by comparing the motion sensor signatures from right and left side ear-wearable devices.

In some embodiments herein, the series of cues (such as audio cues) are delivered differentially through the ear-wearable device 100 and the second ear-wearable device 502. For example, cues for a right step can be delivered through only the ear-wearable device on the right side while cues for a left step can be delivered only through the ear-wearable device on the left side. In some embodiments, cues for a right step can be delivered through the ear-wearable device on the right side at a higher volume while cues for a left step can be delivered through the ear-wearable device on the left side at a higher volume. In some embodiments, cues delivered through ear-wearable device 100 and the second ear-wearable device 502 can sound the same. In other embodiments, cues delivered through ear-wearable device 100 and the second ear-wearable device 502 can sound different, such as being different in volume, pitch, content, and the like.

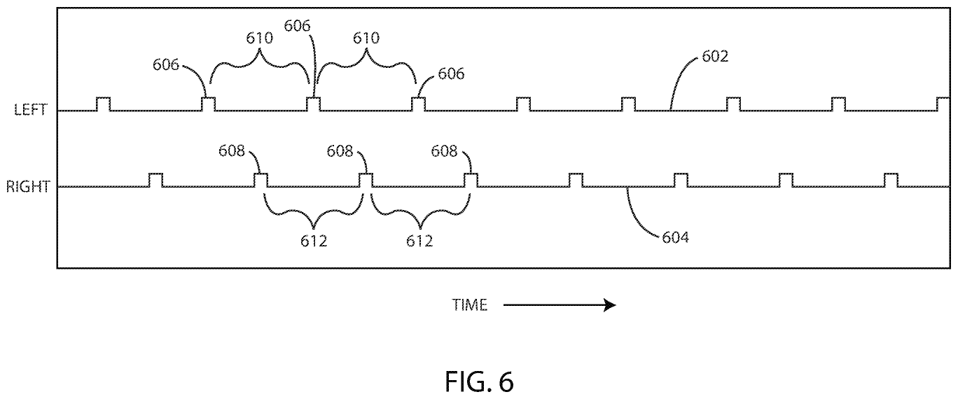

Referring now to FIG. 6, a schematic view of left and right-side steps is shown in accordance with various embodiments herein. This view shows left side gait activity 602 including left-side steps 606. The left side gait activity 602 includes a left step interval 610 between successive left-side steps 606. This view also shows right side gait activity 604 including right side step 608. The right side gait activity 604 includes a right step interval 612 between successive right-side steps 608. As previously discussed, in various embodiments the ear-wearable device 100 can be configured to record signals from at least one sensor, such as at least one of the motion sensor and the microphone, and process the signals to characterize an existing gait of the device wearer 302, including left side gait activity 602 and right side gait activity 604.

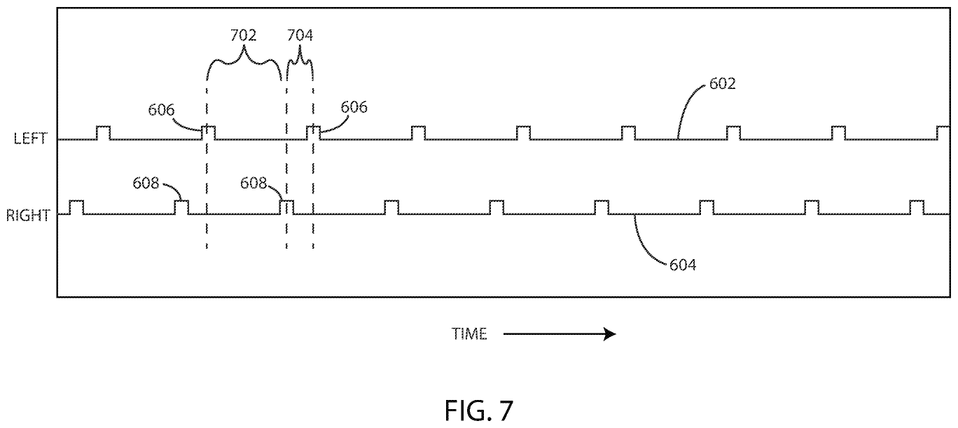

Referring now to FIG. 7, a schematic view of left and right-side steps is shown in accordance with various embodiments herein. FIG. 7 is generally similar to FIG. 6. However, FIG. 7 shows a right side step duration 702 and a left side step duration 704. In this example, it can be seen that the right side step duration 702 is longer than the left side step duration 704. As such, this serves as simply one example of left-right gait asymmetry herein. Other types of left-right gait asymmetry include where left and right foot falls are different in terms of sound, motion sensor signals, and the like.

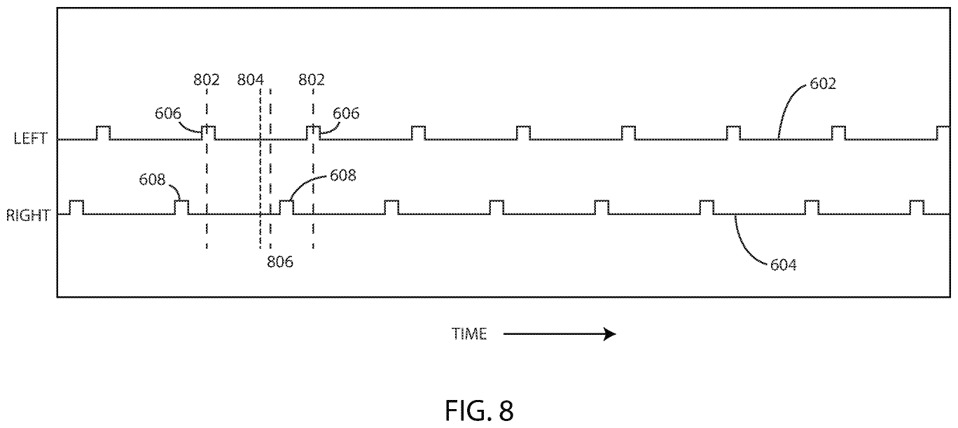

Referring now to FIG. 8, a schematic view of left and right-side steps is shown in accordance with various embodiments herein. FIG. 8 is generally similar to FIGS. 6 and 7. In this type of scenario, the goal may be to cause the gait of the device wearer to be more left-right symmetrical. FIG. 8 shows left side cues 802. FIG. 8 also shows a right target 804 that reflects a fully symmetrical gait. The right side gait activity 604 shown also includes a right cue 806.

In some scenarios, the right cue 806 can simply be provided at a time equal to the right target 804. However, it will be appreciated that in some scenarios it can be beneficial to gradually nudge the device wearer towards a more symmetrical gait without pushing an abrupt change on them. In some examples, a device and/or system herein can deliver cues designed to shape or otherwise influence a wearer's gait, e.g., to treat an unbalanced gait, or speed up a gait, or make a gait more consistent, by matching a cue scheme to the wearers existing gait (e.g., as learned from data) and slowly changing the cue scheme (over minutes, hours, days, or weeks) to lead the wearer through a transition to a more preferred gait.

In the example of FIG. 8, the right cue 806 is disposed between the right target 804 and the right side step 608. As the device wearer, under the influence of the provided cue, shifts their right side step towards or in line with the right cue (over successive strides) the device wearer's gait becomes more symmetrical. Over time, the right cue 806 can be moved closer to the right target 804, or even coincident with the right target 804. In various embodiments, the series of audio cues exhibit a left-right asymmetry that can be characteristic for the device wearer.

In various embodiments, the series of audio cues are configured to reflect an idealized gait specific for the device wearer. In some embodiments, the idealized gait can be input into the device or system by a third party, such as a care provider or a clinician.

In some embodiments, the ear-wearable device can be configured to calculate one or more desired gait parameters and provide a series of audio cues to a device wearer consistent with the one or more desired gait parameters. In various embodiments, the one or more desired gait parameters includes one or more of a gait tempo, a gait cadence, and a left vs. right symmetry value. In various embodiments, the ear-wearable device can be configured to calculate the one or more desired gait parameters by evaluating signals from the motion sensor and/or the microphone and referencing stored data regarding target gait parameters. In various embodiments, the target gait parameters are input by the device wearer, a care provider, or a medical professional. In various embodiments, the ear-wearable device can be configured to calculate the one or more desired gait parameters by evaluating signals from the motion sensor and/or the microphone reflecting a current gait of the device wearer during an observation period.

In some embodiments, the ear-wearable device and/or system herein can also detect posture associated with gait. For example, if the device wearer is leaning too far (forward, backward, or to the side) while walking, this may negatively impact gait as well as generate an elevated fall risk. Posture can be detected using various sensors herein including, for example, an accelerometer that may be part of a motion sensor herein. In some embodiments, sensors herein can also include a gyroscope that can be used to detect angular deviations associated with gait, such as leaning forward. In various embodiments, the device and/or system can be configured to instruct the device wearer regarding a proper posture if an abnormal posture or angular deviation is detected while walking.

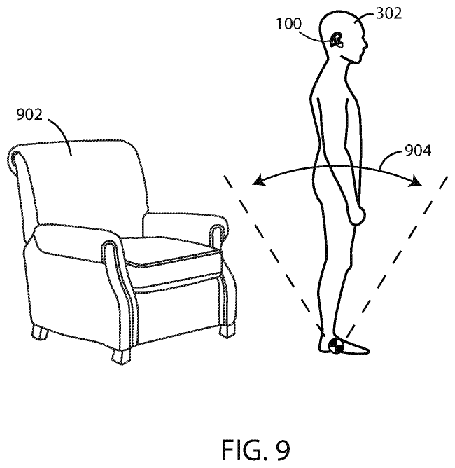

Referring now to FIG. 9, a schematic view of a device wearer 302 getting up from a chair 902 is shown in accordance with various embodiments herein. FIG. 9 also shows the device wearer 302 with an ear-wearable device 100. The device wearer 302 is illustrated exhibiting a degree of sway 904. Sway 904 can be measured using signals from a motion sensor. The magnitude of sway 904 can be evaluated by the ear-wearable device.

In various embodiments, the ear-wearable device 100 can be configured to initiate or discontinue the series of audio cues based on a detected activity state as reflected in data from the motion sensor and/or the microphone. In various embodiments, the detected activity state can include the device wearer 302 assuming a standing or upright posture. In various embodiments, the detected activity state can include cessation of the device wearer 302 walking.

In some embodiments herein, a device and/or system can be configured to determine (e.g., using sensor data, such as a motion sensor) that a wearer is walking, or is about to walk (e.g., after a sit-to-stand transition) and begin to deliver auditory cues to support the wearer while walking. The system can also determine (e.g., using motion sensor data) that a wearer has stopped walking (e.g., has reached a destination or stopped to participate in a conversation) and end an auditory cue scheme when the walking has stopped.

In some embodiments herein, a device and/or system can be configured to detect a sit-to-stand transition (and/or the rapidity of the same) and start to deliver gait cues after a standing or upright posture has been reached. However, an excessively fast transition can result in the device wearer becoming unstable. As such, in some embodiments, if the rapidity of the sit-to-stand transition crosses a threshold value, the device or system can wait before beginning to provide gait cues.

In some embodiments, the device or system herein can use information such as information regarding the environment that the device wearer is in to initiate, terminate, and/or modulate the gait therapy provided herein. Referring now to FIG. 10, a schematic view of environments is shown in accordance with various embodiments herein. FIG. 10 shows a device wearer 302. In this view, the device wearer 302 is currently in an indoor environment 1002. FIG. 10 also shows an outdoor environment 1004. Sound field properties such as echoes, reverberation time, decay time, critical distance, room impulse measure, absorption coefficient across a human detectable frequency band, ambient noise, comb filter distortion, coloration distortion, early reflection, and late reflection, and the like can vary with different sound fields. As such, by evaluating such properties, the sound field (or environment) can be characterized. In some embodiments, an indoor environment 1002 can be distinguished from an outdoor environment 1004 as an indoor environment 1002 is more likely to exhibit significant echoes. In some embodiments, the device can actively identify the environment, such as by emitting a test tone and then monitoring for the response. In other embodiments, the device can passively identify the environment by monitoring sounds as picked up by a microphone. In some embodiments, herein, the device can identify an environment, such as the device wearer 302 being in an outdoor environment 1004, and then initiate gait therapy as described herein. In some embodiments, herein, the device can first identify an activity, such as walking, then identify an environment, such as the device wearer 302 being in an outdoor environment 1004, and then initiate gait therapy as described herein if the device wearer 302 is, for example, determined to be walking in an outdoor environment 1004. As such, in various embodiments herein, initiation, termination, or modulation of gait therapy herein can take into consideration at least one of activity of the device wearer and the environment of the device wearer.

In some embodiments, locations and/or environments can be determined by the device or system and recorded along with gait data. This can allow a determination of the impact of specific locations on gait. Location can be determined in various ways. In some embodiments, location can be determined using a GPS signal or a similar geolocation signal. Similarly, in some embodiments, the ear-wearable device 100 can be configured to record identifying wireless packets (such as advertising packets) encountered and cross-reference gait against the recorded identifying wireless packets.

Identifying wireless packets can, in some cases, also be used for contact tracing. By way of example, if the device or system detected wireless packets coming from a device that is personal to another individual, such as a smartphone, then the detection of those packets can be used as a proxy for the presence of the individual. Also, in some embodiments, the device or system herein can use sensor data, such as data from a microphone, in order to detect the unique signature of the voice of another individual. In various embodiments, data regarding other individuals that are present can be recorded in order to determine their effect on the gait (and elements of gait such as tempo) of the device wearer. In some embodiments, the ear-wearable device 100 can use information regarding other detected individuals in setting parameters of gait training or therapy. By way of example, if the device or system learns that gait tempo is always faster than normal when a given individual is present, then the system can initiate gait training or therapy at a commensurately faster pace when that individual or a proxy thereof is detected.

In various embodiments herein, the time of day (and/or time of week or month) can be evaluated as part of characterizing the gait of the device wearer and/or in providing gait training or therapy. For example, in various embodiments herein, the time of day can be stored along with data regarding the gait. Then, using such data, patterns regarding the relationship of time and gait can be derived regarding the pattern recognition and/or matching techniques described herein. For example, for a given device wearer, the device or system can detect that walks taken in the morning tend to be at a higher tempo or cadence. Such information can then be used by the device or system. For example, in that same example, if gait training or therapy is initiated during the morning, then the system can initiate it at a higher pace or tempo. Conversely, if walks for a given individual tend to be slower during the evening hours, then the device or system can initiate gait training or therapy at a slower pace or tempo during the evening hours. Also, detected patterns regarding gait and parameters of gait can be stored and/or reported to a third party, such as a care provider or a clinician to provide them with more insight regarding the current status of the device wearer.

In various embodiments, the ear-wearable device 100 can be configured to detect whether a device wearer is changing elevation and initiate, terminate, or modulate the gait therapy. By way of example, in some embodiments, the ear-wearable device 100 can be configured to detect whether a device wearer is changing elevation and change a tempo of the series of audio cues accordingly. For example, the device wearer could be changing elevation by going up or down stairs or walking up or down a hill. In such a case, the device can be configured to terminate gait therapy, pause gait therapy, and/or reduce the tempo of gait therapy.

It can be desirable to modulate gait therapy based on ambient conditions. For example, if the temperature is high and/or the humidity is high, it may be better for the device wearer if gait therapy is modulated accordingly. For example, if temperature and/or humidity crosses a threshold value (which can be determined based on sensors associated with the device and/or receiving data from a weather API), then the gait therapy can be modulated, such as by reducing a tempo thereof. In some embodiments, the device and/or system can be configured to not initiate gait training therapy if environmental conditions make it unsafe. By way of example, if snow or ice is detected for the location of the device wearer (such as could be determined using a weather API), then the device and/or system can be configured to refrain from initiating gait training or therapy.

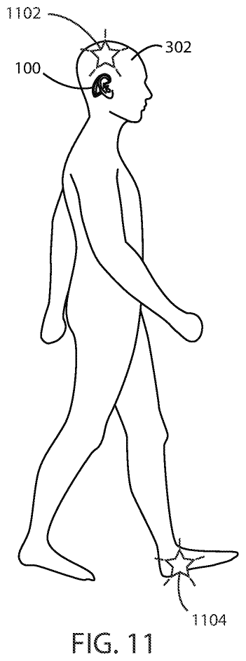

Referring now to FIG. 11, a schematic view is shown of a device wearer 302 with an ear-wearable device 100 illustrating characterization of a current gait to detect a health status, an injury, or another condition. The device wearer 302 could have an injury or a condition. For example, the device wearer 302 could have a neurological injury 1102, which could be acute or chronic, or could specifically be a stroke, a traumatic brain injury, or the like. Such neurological injuries 1102 can be reflected in the gait of the device wearer 302. As such, analysis of the device wearer's 302 gait can be used to identify and/or evaluate a neurological injury 1102 or condition.

Similarly, the device wearer 302 could have a musculoskeletal injury 1104. It will be appreciated that there are many different musculoskeletal injuries 1104 that can be reflected in the gait of the device wearer 302 including, but not limited to, foot injuries, ankle injuries, leg injuries, knee injuries, hip injuries, back injuries, and the like. As such, analysis of the device wearer's 302 gait can be used to identify and/or evaluate a musculoskeletal injury 1104 or condition.

In various embodiments, the ear-wearable device 100 can be configured to match a set of data (such as data from the sensors herein) against a plurality of predetermined patterns to characterize the current gait. In various embodiments, the ear-wearable device 100 can be configured to match the set of data against a plurality of predetermined patterns to characterize a current health status of a device wearer. In various embodiments, the ear-wearable device 100 can be configured to determine whether the characterized current gait reflects a neurological injury and, in some cases, whether the injury or condition appears to be acute or chronic based on how suddenly the pattern has emerged in the device wearer's gait. In various embodiments, the ear-wearable device 100 can be configured to determine whether the characterized current gait reflects a musculoskeletal injury or imbalance. In various embodiments, the ear-wearable device 100 can be configured to alert a device wearer and/or a third party regarding a possible injury being detected. In various embodiments, the ear-wearable device 100 can be configured to alert a device wearer and/or a third party regarding a possible injury being detected along with a recommendation to prevent further injury, such as ceasing a current activity. In some embodiments, such as where a gait pattern has been identified that matches a gait pattern associated with neurological injuries, the device can issue an alert for a coach, referee or other responsible party that an athlete (as a device wearer) may have suffered a neurological injury such as a traumatic brain injury based on the pattern of their gait. In some embodiments, such as where a gait pattern has been identified that matches a gait pattern associated with injuries, the device can issue an alert for a supervisor, military officer, or other responsible party that a soldier may have suffered an injury based on the pattern of their gait.

In various embodiments, the ear-wearable device 100 can be configured to generate a suggestion regarding a physical activity to ameliorate the musculoskeletal injury or imbalance. For example, if the device determines that the device wearer suffers from a right-side weakness, the device can generate a suggestion regarding a physical activity to strengthen the right side. As another example, if the device determines that a particular muscle or muscle group of the device wearer is weak, based on their gait machine a pattern indicative of such a weakness, the device and/or system can generate a suggestion regarding an activity to strengthen the particular muscle or muscle group.

In various embodiments, the ear-wearable device 100 can be configured to determine whether a change from the previous gait to the current gait reflects an injury. In various embodiments, the ear-wearable device 100 can be configured to determine whether the change from the previous gait to the current gait reflects a neurological disease state or a neurological injury. In various embodiments, the ear-wearable device 100 can be configured to determine whether the change from the previous gait to the current gait reflects a musculoskeletal injury.

In various embodiments, the ear-wearable device 100 can be configured to identify a condition of hypokinetic feet (such as may occur with Parkinson's disease) based on the characterization of the current gait. Hypokinetic feet can include the device wearer being hunched over and taking little steps before they start moving, sometimes also associated with a tremor. In some embodiments, the device wearer may take tiny steps sideways (e.g., move in circle) until pointed in the direction they want to go. Detection of these events can indicate an intent to start walking. In some embodiments herein, the ear-wearable device can start delivering auditory cue in response to detection of an intent to start walking.

In some embodiments herein, the ear-wearable device 100 can be configured to match a set of data (such as data from the sensors herein) against one or more predetermined patterns that serve as positive examples or negative examples of hypokinetic feet (or another condition) to determine whether a condition of hypokinetic feet is present or not.

In various embodiments herein, the ear-wearable device 100 can be configured to characterize a current emotional status of a device wearer based on the current gait of the device wearer. For example, the ear-wearable device 100 can be configured to match a set of data (such as data from the sensors herein) against a plurality of predetermined patterns that are associated with emotional states (angry, stressed, depressed, etc.) to characterize the current emotional status of the device wearer.

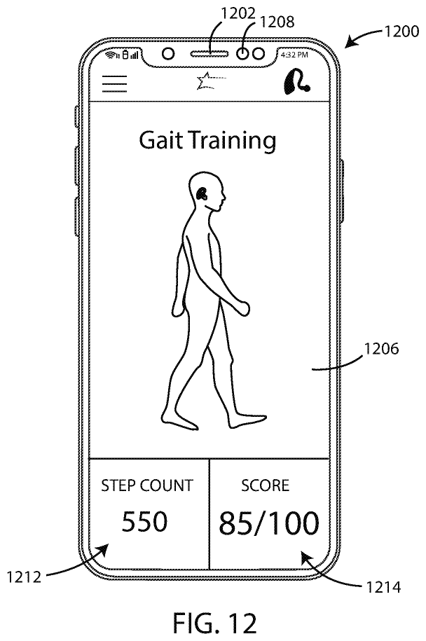

In various embodiments herein, device can interface with an accessory device and/or systems herein can include an accessory device. Referring now to FIG. 12, a schematic view of an accessory device 1200 is shown in accordance with various embodiments herein. The accessory device 1200 can include a display screen 1206 thereof. The accessory device 1200 can also include a speaker 1202 and a front-facing camera 1208.

In various embodiments, information regarding gait training can be displayed on the display screen 1206. By way of example, in some embodiments, a step count 1212 can be displayed. In some embodiments, a score 1214 can be displayed, wherein the score can reflect various aspects such as the symmetry of gait achieved by the device wearer, how well the device wearer is following cues, etc. Other pieces of information can also be displayed including, for example, an elapsed time for a current gait therapy session, an amount of time remaining for a current gait therapy session, historical information on past gait therapy sessions, and the like. In some embodiments, such pieces of information can also be audio streamed to the ear-wearable device(s).

In some embodiments, the display screen 1206 can display a virtual reality image, icon, or avatar illustrated to be walking as the device wearer takes steps. The image, icon, or avatar can show the correct motion to encourage the device wearer to mimic that motion by taking steps and/or changing their gait. In various embodiments, the device can send commands and/or data to the accessory device 1200 to facilitate such functionality. In some embodiments, the accessory device 1200 can operate a game wherein the device wearer gets points when they successfully complete actions as illustrated by the image, icon or avatar and/or complete actions with the same or similar gait as illustrated.

Gait asymmetry herein can be evaluated in various ways. In some embodiments, gait asymmetry can be comparing one or more left side gait parameters with one or more corresponding right side gait parameters. As a simple example, the sound volume of left-side steps can be compared with the sound volume of right-side steps and gait asymmetry can be calculated as an average percentage difference reflecting decibels between left and right steps. As another example, the magnitude of motion sensor signals of left-side steps can be compared with the magnitude of motion sensor signals of right-side steps and gait asymmetry can be calculated as an average percentage difference (or another statistical measure) reflecting the difference between left and right steps. As another example, the step timing of left-side steps can be compared with the step timing of right-side steps and gait asymmetry can be calculated as a comparison between left and right steps. As yet another example, by evaluating motion sensor signals and, in some cases, positional or geolocation data, stride lengths can be estimated, and the estimated stride length of left-side steps can be compared with the estimated stride length of right-side steps and gait asymmetry can be calculated as a comparison between left and right stride lengths. Many other techniques of calculating a value and statistics for gait asymmetry are also contemplated herein.

In various embodiments, the ear-wearable device 100 can be configured to interface with an accessory device and send or receive information regarding one or more current and/or target or desired gait parameters.

In various embodiments, the ear-wearable device 100 can be configured to detect an elevated fall risk based on the device wearer's gait. For example, the ear-wearable device 100 can detect an elevated fall risk if the device wearer's gait matches a pattern associated with an elevated fall risk. In various embodiments, the ear-wearable device 100 can be configured to send a notification and/or a control signal to an accessory device and/or secondary device when an elevated fall risk is be present. In various embodiments, the ear-wearable device 100 can be configured to send instructions to a device wearer (or another individual) when an elevated fall risk is present to reduce or mitigate the risk of a fall. For example, the ear-wearable device 100 can send an instruction to the device wearer to sit down, pause, and/or use an assistive walking device.

In various embodiments, the accessory device and/or secondary device can include one with home automation features. By way of example, in some embodiments, the accessory device and/or secondary device can include one with an ability to control lights. In some embodiments, the ear-wearable device 100 can send home automation commands when an elevated fall risk is detected through characterization of the device wearer's gait. For example, the ear-wearable device 100 can send a command to a home automation system to turn on lights to make a fall less likely (such as if the device wearer gets up in the night and is unsteady).

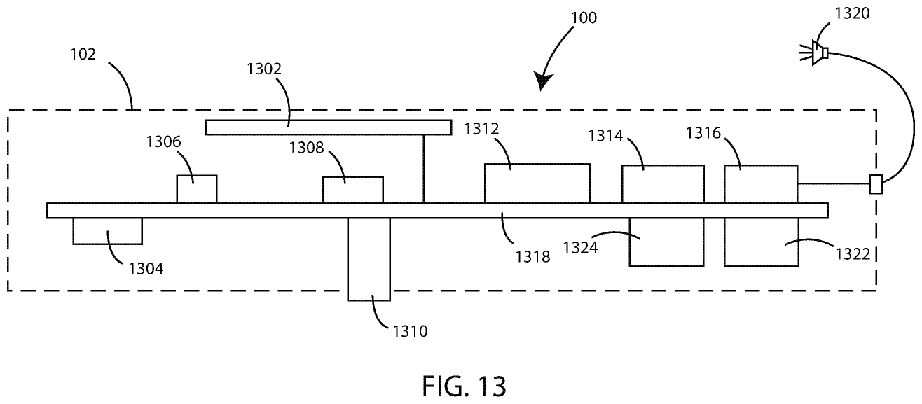

Referring now to FIG. 13, a schematic block diagram is shown illustrating various components of an ear-wearable device in accordance with various embodiments herein. It will be appreciated that many of these components can be integrated in an integrated circuit, such as with a system-on-a-chip (SOC) integration or can exist as separate components. The block diagram of FIG. 13 represents a generic ear-wearable device for purposes of illustration. The ear-wearable device 100 shown in FIG. 13 includes several components electrically connected to a flexible mother circuit 1318 (e.g., flexible mother board) which is disposed within housing 102. A power supply circuit 1304 can include a battery and can be electrically connected to the flexible mother circuit 1318 and provides power to the various components of the ear-wearable device 100. One or more microphones 1306 are electrically connected to the flexible mother circuit 1318, which provides electrical communication between the microphones 1306 and a digital signal processor (DSP) 1312. Among other components, the DSP 1312 incorporates or is coupled to audio signal processing circuitry configured to implement various functions described herein. A sensor package 1314 can be coupled to the DSP 1312 via the flexible mother circuit 1318. The sensor package 1314 can include one or more different specific types of sensors such as those described in greater detail below. One or more user switches 1310 (e.g., on/off, volume, mic directional settings) are electrically coupled to the DSP 1312 via the flexible mother circuit 1318.

An audio output device 1316 is electrically connected to the DSP 1312 via the flexible mother circuit 1318. In some embodiments, the audio output device 1316 comprises a speaker (coupled to an amplifier). In other embodiments, the audio output device 1316 comprises an amplifier coupled to an external receiver 1320 adapted for positioning within an ear of a wearer. The external receiver 1320 can include an electroacoustic transducer, speaker, or loudspeaker. The ear-wearable device 100 may incorporate a communication device 1308 coupled to the flexible mother circuit 1318 and to an antenna 1302 directly or indirectly via the flexible mother circuit 1318. The communication device 1308 can be a Bluetooth® transceiver, such as a BLE (Bluetooth® low energy) transceiver or other transceiver(s) (e.g., an IEEE 802.11 compliant device). The communication device 1308 can be configured to communicate with one or more external devices, such as those discussed previously, in accordance with various embodiments. In various embodiments, the communication device 1308 can be configured to communicate with an external visual display device such as a smart phone, a video display screen, a tablet, a computer, a television, a virtual or augmented reality, a hologram, or the like.

In various embodiments, the ear-wearable device 100 can also include a control circuit 1322 and a memory storage device 1324. The control circuit 1322 can be in electrical communication with other components of the device. The control circuit 1322 can execute various operations, such as those described herein. The control circuit 1322 can include various components including, but not limited to, a microprocessor, a microcontroller, an FPGA (field-programmable gate array) processing device, an ASIC (application specific integrated circuit), or the like. The memory storage device 1324 can include both volatile and non-volatile memory. The memory storage device 1324 can include ROM, RAM, flash memory, EEPROM, SSD devices, NAND chips, and the like. The memory storage device 1324 can be used to store data from sensors as described herein and/or processed data generated using data from sensors as described herein.

In various embodiments, a spatial location determining circuit (or geolocation circuit) can be included and can take the form of an integrated circuit that can include components for receiving signals from GPS, GLONASS, BeiDou, Galileo, SBAS, WLAN, BT, FM, and/or NFC type protocols.

### Methods

Many different methods are contemplated herein, including, but not limited to, a method of providing gait training or therapy to a device wearer. The method can include
calculating one or more desired gait parameters and providing a series of audio cues to a device wearer consistent with the one or more desired gait parameters.

In another embodiment, a method or providing gait training or therapy can include operating in a first mode, wherein the first mode includes evaluating signals from at least one of the motion sensor and the microphone to characterize a gait of a device wearer, and operating in a second mode, wherein the second mode includes providing a series of audio cues to the device wearer.

In another embodiment, a method or providing gait training or therapy can include generating a set of data reflecting a current gait of a device wearer based on signals from at least one of the motion sensor and the microphone and matching the set of data against a plurality of predetermined patterns to characterize the current gait.

In another embodiment, a method or providing gait training or therapy can include generating a set of data reflecting a current gait of a device wearer based on signals from at least one of the motion sensor and the microphone, comparing the set of data against stored data reflecting a previous gait of the device wearer, and characterizing a health status of the device wearer based on a change from the previous gait to the current gait of the device wearer.

In various embodiments herein, methods can include one or more operations of calculating one or more desired gait parameters, providing a series of audio cues to a device wearer consistent with the one or more desired gait parameters, recording signals from at least one of the motion sensor and the microphone and process the signals to characterize an existing gait of the device wearer, calculating the one or more desired gait parameters by evaluating signals from the motion sensor and/or the microphone and referencing stored data regarding target gait parameters, calculating the one or more desired gait parameters by evaluating signals from the motion sensor and/or the microphone reflecting a current gait of the device wearer during an observation period, normalizing the one or more desired gait parameters based on a detected activity level as reflected in data from the motion sensor, initiating or discontinue the series of audio cues based on a detected activity state as reflected in data from the motion sensor and/or the microphone, initiating the series of audio cues based on detection of an abnormal or atypical gait, initiating the series of audio cues based on detection of a gait with a step timing variability or statistics crossing a threshold value, initiating the series of audio cues based on detection of a gait with a left-right symmetry variability or statistics crossing a threshold value, initiating the series of audio cues based on detection of the presence of a device wearer within a particular environment, evaluating data from the motion sensor over a time period to determine a range or statistics relating to gait tempo values for the device wearer, adjusting a volume, frequency specific amplification, frequency shifting, frequency compression, and/or frequency transposition of the series of audio cues, evaluating a response of the device wearer in response to the series of audio cues evaluating a response of the device wearer in response to the series of audio cues and adjust the series of audio cues accordingly, distinguishing between a right step and a left step based on an input received from an accessory device, characterizing a gait of the device wearer at varying levels of physical exertion, characterizing a gait of the device wearer at varying levels of physical exertion, detect whether the device wearer is changing elevation and change one or more properties of the series of audio cues accordingly, sensing ambient conditions around the device wearer change a tempo of the series of audio cues accordingly, recording identifying wireless packets encountered and cross-reference gait against the recorded identifying wireless packets, generating a set of data reflecting a current gait of a device wearer based on signals from at least one of the motion sensor and the microphone, matching the set of data against a plurality of predetermined patterns to characterize the current gait, matching the set of data against a plurality of predetermined patterns to characterize a current health status of the device wearer, generating a set of data reflecting a current gait of a device wearer based on signals from at least one of the motion sensor and the microphone, comparing the set of data against stored data reflecting a previous gait of the device wearer, and/or characterizing a health status of the device wearer based on a change from the previous gait to the current gait of the device wearer.

Aspects of system/device operation described elsewhere herein can be performed as operations of one or more methods in accordance with various embodiments herein.

### Pattern Identification and Matching

It will be appreciated that in various embodiments herein, a device or a system can be used to detect a gait pattern or patterns indicative of a type of gait, a health status or condition, a neurological injury or condition, a musculoskeletal injury or condition, or the like. Such patterns can be detected in various ways. Some techniques are described elsewhere herein, but some further examples will now be described.

As merely one example, one or more sensors can be operatively connected to a controller (such as the control circuit described in FIG. 13) or another processing resource (such as a processor of another device or a processing resource in the cloud). The controller or other processing resource can be adapted to receive data representative of a gait of the device wearer from one or more of the sensors and/or determine gait statistics of the subject over a monitoring time period based upon the data received from the sensor(s). As used herein, the term “data” can include a single datum or a plurality of data values or statistics. The term “statistics” can include any appropriate mathematical calculation or metric relative to data interpretation, e.g., probability, confidence interval, distribution, range, or the like. Further, as used herein, the term “monitoring time period” means a period of time over which characteristics of the subject are measured and statistics are determined. The monitoring time period can be any suitable length of time, e.g., 1 millisecond, 1 second, 10 seconds, 30 seconds, 1 minute, 10 minutes, 30 minutes, 1 hour, etc., or a range of time between any of the foregoing time periods.

Any suitable technique or techniques can be utilized to determine statistics for the various data from the sensors, e.g., direct statistical analyses of time series data from the sensors, differential statistics, comparisons to baseline or statistical models of similar data, etc. Such techniques can be general or individual-specific and represent long-term or short-term behavior. These techniques could include standard pattern classification methods such as Gaussian mixture models, clustering as well as Bayesian approaches, machine learning approaches such as neural network models and deep learning, and the like.

Further, in some embodiments, the controller can be adapted to compare data, data features, and/or statistics against various other patterns, which could be prerecorded gait patterns (baseline patterns) of the particular individual wearing an ear-wearable device herein, prerecorded gait patterns (group baseline patterns) of a group of individuals wearing ear-wearable devices herein, one or more predetermined gait patterns that serve as patterns indicative of an occurrence of a particular health status/event, injury or condition (positive example patterns), one or more predetermined gait patterns that serve as patterns indicative of the absence of a particular health status/event, injury or condition (negative example patterns), or the like. As merely one scenario, if a gait pattern is detected in an individual that exhibits similarity crossing a threshold value to a particular positive example pattern or substantial similarity to that pattern, wherein the pattern is specific for a particular health status/event, injury or condition, then that can be taken as an indication of an occurrence of a particular health status/event, injury or condition.

Similarity and dissimilarity can be measured directly via standard statistical metrics such normalized Z-score, or similar multidimensional distance measures (e.g., Mahalanobis or Bhattacharyya distance metrics), or through similarities of modeled data and machine learning. These techniques can include standard pattern classification methods such as Gaussian mixture models, clustering as well as Bayesian approaches, neural network models, and deep learning.

As used herein the term “substantially similar” means that, upon comparison, the sensor data are congruent or have statistics fitting the same statistical model, each with an acceptable degree of confidence. The threshold for the acceptability of a confidence statistic may vary depending upon the subject, sensor, sensor arrangement, type of data, context, condition, etc.

The statistics associated with the gait of an individual over the monitoring time period can be determined by utilizing any suitable technique or techniques, e.g., standard pattern classification methods such as Gaussian mixture models, clustering, hidden Markov models, as well as Bayesian approaches, neural network models, and deep learning.

Various embodiments herein specifically include the application of a machine learning classification model. In various embodiments, the ear-wearable devices and/or systems herein can be configured to periodically update the machine learning classification model based on gait of the device wearer.

In some embodiments, a training set of data can be used in order to generate a machine learning classification model. The input data can include microphone and/or sensor data as described herein as tagged/labeled with binary and/or non-binary classifications of gait or elements of gait. Binary classification approaches can utilize techniques including, but not limited to, logistic regression, k-nearest neighbors, decision trees, support vector machine approaches, naive Bayes techniques, and the like. Multi-class classification approaches (e.g., for non-binary classifications of gait) can include k-nearest neighbors, decision trees, naive Bayes approaches, random forest approaches, and gradient boosting approaches amongst others.

In some embodiments, to facilitate a supervised machine learning approach, a device wearer can be put through a particular movement protocol (such as a particular walking protocol) in order to provide a training set of data that is specific for the device wearer. In some embodiments, a training set of data specific for the device wearer can be gathered as part of a fitting procedure associated with the device wearer getting the device(s). However, in other embodiments, unsupervised machine learning approaches can also be used.

In various embodiments, the device and/or system herein is configured to execute operations to generate or update the machine learning model on the ear-wearable device itself. In some embodiments, the ear-wearable device may convey data to another device such as an accessory device or a cloud computing resource in order to execute operations to generate or update a machine learning model herein.

In various embodiments herein, threshold values used herein (as described at various points herein) can be calculated or otherwise derived through analysis of data regarding the device wearer. For example, in some embodiments, a threshold value can be set through evaluation of previous events related to the gait of the device wearer. The events can include, but are not limited to, one or more of gait freezes, stumbles, falls, cessation of walking, or continued walking with appreciably the same gait metrics or improved. In some cases, such events can be detected by the ear-wearable device(s). In other cases, such events can be provided as input to the ear-wearable device(s) from another system, device, or third party. In some embodiments, the threshold value can be related to the occurrence of such events. In some embodiments, the threshold value can be related to the prediction of the occurrence of such events based on a comparison of past gait data associated with the occurrence of such events and current gait data. In some embodiments, the threshold value can be related to a characterization of the device wearer's gait associated with the occurrence of such events. In some embodiments, the threshold value can divide categories of relevance for gait training such that a process of categorization also calculates threshold value(s). Categorization and/or calculation of threshold values can, in some cases, be performed using a machine learning approach including for example, an unsupervised machine learning approach. However, in some scenarios, supervised machine learning approaches can also be used. In some embodiments, calculation of threshold values can be performed using statistical approaches.

### Virtual Spatialized Sound

In some embodiments, a series of audio cues herein can take the form of virtual spatialized sounds (e.g., sounds delivered in a manner to provide the perception of having a specific spatial origin), such as a cue delivered to be perceived as originating on the left-side related to a left-side step followed by a cue delivered to be perceived as originating on the right-side related to a right-side step and so on. As another example, the device and/or system can be configured to present virtualized auditory objects for the device wearer to try to step on (to help coordinate stride lengths and timing). Virtual spatialized audio can be generated by applying a head-related transfer function (HRTF) filter to the audio stream or channel. Details of virtual spatialized audio are provided in U.S. Publ. Appl. No. 2018/0317837 and U.S. Pat. No. 9,848,273, the content of both of which is herein incorporated by reference.

### Sensor Package

Various embodiments herein include one or more sensors. Specifically, devices and systems herein can include one or more sensors (including one or more discrete or integrated sensors) to provide data for use with operations to evaluate and/or characterize the gait of a device wearer. Further details about the sensors are provided as follows. However, it will be appreciated that this is merely provided by way of example and that further variations are contemplated herein. Also, it will be appreciated that a single sensor may provide more than one type of physiological data. For example, heart rate, respiration, blood pressure, or any combination thereof may be extracted from PPG (photoplethysmography) sensor data.

In various embodiments, the gait of the device wearer is characterized using data produced by at least one of the motion sensor and the microphone. In various embodiments, other sensors can also be included such as at least one of a heart rate sensor, a heart rate variability sensor, an electrocardiogram (ECG) sensor, a blood oxygen sensor, a blood pressure sensor, a skin conductance sensor, a photoplethysmography (PPG) sensor, a temperature sensor (such as a core body temperature sensor, skin temperature sensor, ear-canal temperature sensor, or another temperature sensor), a motion sensor, an electroencephalograph (EEG) sensor, and a respiratory sensor. In various embodiments, the motion sensor can include at least one of an accelerometer and a gyroscope.

Devices herein can specifically include one or more motion sensors (or movement sensors) amongst other types of sensors. Motion sensors herein can include inertial measurement units (IMU), accelerometers, gyroscopes, barometers, altimeters, and the like. The IMU can be of a type disclosed in commonly owned U.S. patent application Ser. No. 15/331,230, filed Oct. 21, 2016, which is incorporated herein by reference. In some embodiments, electromagnetic communication radios or electromagnetic field sensors (e.g., telecoil, NFMI, TMR, GMR, etc.) sensors may be used to detect motion or changes in position. In some embodiments, biometric sensors may be used to detect body motions or physical activity. Motion sensors can be used to track movements of a patient in accordance with various embodiments herein.

In some embodiments, the motion sensors can be disposed in a fixed position with respect to the head of a patient, such as worn on or near the head or ears. In some embodiments, operatively connected motion sensors can be worn on or near another part of the body such as on a wrist, arm, or leg of the patient.

According to various embodiments, sensors herein can include one or more of an IMU, and accelerometer (3, 6, or 9 axis), a gyroscope, a barometer, an altimeter, a magnetometer, a magnetic sensor, an eye movement sensor, a pressure sensor, an acoustic sensor, a telecoil, a heart rate sensor, a global positioning system (GPS) circuit, a temperature sensor, a blood pressure sensor, an oxygen saturation sensor, an optical sensor, a blood glucose sensor (optical or otherwise), a galvanic skin response sensor, a cortisol level sensor (optical or otherwise), a microphone, acoustic sensor, an electrocardiogram (ECG) sensor, electroencephalography (EEG) sensor which can be a neurological sensor, eye movement sensor (e.g., electrooculogram (EOG) sensor), myographic potential electrode sensor (or electromyography—EMG), a heart rate monitor, a pulse oximeter or oxygen saturation sensor (SpO2), a wireless radio antenna, blood perfusion sensor, hydrometer, sweat sensor, cerumen sensor, air quality sensor, pupillometry sensor, cortisol level sensor, hematocrit sensor, light sensor, image sensor, and the like.

In some embodiments, sensors herein can be part of an ear-wearable device. However, in some embodiments, the sensors utilized can include one or more additional sensors that are external to an ear-wearable device. For example, various of the sensors described above can be part of a wrist-worn or ankle-worn sensor package, or a sensor package supported by a chest strap. In some embodiments, sensors herein can be disposable sensors that are adhered to the device wearer (“adhesive sensors”) and that provide data to the ear-wearable device or another component of the system.

Data produced by the sensor(s) herein can be operated on by a processor of the device or system.

As used herein the term “inertial measurement unit” or “IMU” shall refer to an electronic device that can generate signals related to a body's specific force and/or angular rate. IMUs herein can include one or more accelerometers (3, 6, or 9 axis) to detect linear acceleration and a gyroscope to detect rotational rate. In some embodiments, an IMU can also include a magnetometer to detect a magnetic field.

As used herein, the term “microphone” shall include reference to all types of devices used to capture sounds including various types of microphones (including, but not limited to, carbon microphones, fiber optic microphones, dynamic microphones, electret microphones, ribbon microphones, laser microphones, condenser microphones, cardioid microphones, crystal microphones) and vibration sensors (including, but not limited to accelerometers and various types of pressure sensors). Microphones herein can include analog and digital microphones. Systems herein can also include various signal processing chips and components such as analog-to-digital converters and digital-to-analog converters. Systems herein can operate with audio data that is gathered, transmitted, and/or processed reflecting various sampling rates. By way of example, sampling rates used herein can include 8,000 Hz, 11,025 Hz, 16,000 Hz, 22,050 Hz, 32,000 Hz, 37,800 Hz, 44,056 Hz, 44,100 Hz, 47,250 Hz, 48,000 Hz, 50,000 Hz, 50,400 Hz, 64,000 Hz, 88,200 Hz, 96,000 Hz, 176,400 Hz, 192,000 Hz, or higher or lower, or within a range falling between any of the foregoing. Audio data herein can reflect various bit depths including, but not limited to 8, 16, and 24-bit depth. Microphones herein can include both directional and omnidirectional microphones. In some embodiments, microphones herein can be configured to be sensitive to sounds coming from the direction of the device wearer's feet to more sensitively pick up the sound of foot falls while walking. In some embodiments, microphones herein can include inward facing microphones to be more sensitive to pickup foot fall sounds through the body.

An eye movement sensor herein may be, for example, an electrooculographic (EOG) sensor, such as an EOG sensor disclosed in commonly owned U.S. Pat. No. 9,167,356, which is incorporated herein by reference. A pressure sensor herein can be, for example, a MEMS-based pressure sensor, a piezo-resistive pressure sensor, a flexion sensor, a strain sensor, a diaphragm-type sensor and the like.

A temperature sensor herein can be, for example, a thermistor (thermally sensitive resistor), a resistance temperature detector, a thermocouple, a semiconductor-based sensor, an infrared sensor, or the like.

A blood pressure sensor herein can be, for example, a pressure sensor. The heart rate sensor can be, for example, an electrical signal sensor, an acoustic sensor, a pressure sensor, an infrared sensor, an optical sensor, or the like.

An oxygen saturation sensor (such as a blood oximetry sensor) herein can be, for example, an optical sensor, an infrared sensor, a visible light sensor, or the like.

An electrical signal sensor herein can include two or more electrodes and can include circuitry to sense and record electrical signals including sensed electrical potentials and the magnitude thereof (according to Ohm's law where V=IR) as well as measure impedance from an applied electrical potential.

It will be appreciated that sensors herein can include one or more sensors that are external to the ear-wearable device. In addition to the external sensors discussed hereinabove, the sensor package can comprise a network of body sensors (such as those listed above) that sense movement of a multiplicity of body parts (e.g., arms, legs, torso). In some embodiments, the ear-wearable device can be in electronic communication with the sensors or processor of a medical device.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. An ear-wearable device comprising:
a control circuit;
a motion sensor, wherein the motion sensor is in electrical communication with the control circuit;
a microphone, wherein the microphone is in electrical communication with the control circuit; and
an electroacoustic transducer, wherein the electroacoustic transducer is in electrical communication with the control circuit;
wherein the ear-wearable device is configured to
calculate one or more desired gait parameters; and
provide a series of audio cues to a device wearer consistent with the one or more desired gait parameters.

2. The ear-wearable device of any of claims 1 and 3-38, wherein the series of audio cues comprise a rhythmic sequence.

3. The ear-wearable device of any of claims 1-2 and 4-38, wherein the series of audio cues comprise speech.

4. The ear-wearable device of any of claims 1-3 and 5-38, wherein the series of audio cues comprise virtual spatialized audio.

5. The ear-wearable device of any of claims 1-4 and 6-38, wherein the one or more desired gait parameters includes one or more of a gait tempo, a gait cadence, step impact magnitude, and a left vs. right symmetry value.

6. The ear-wearable device of any of claims 1-5 and 7-38, wherein the ear-wearable device is configured to record signals from at least one of the motion sensor and the microphone and process the signals to characterize an existing gait of the device wearer.

7. The ear-wearable device of any of claims 1-6 and 8-38, wherein the ear-wearable device is configured to calculate the one or more desired gait parameters by evaluating signals from the motion sensor and/or the microphone and referencing stored data regarding target gait parameters.

8. The ear-wearable device of any of claims 1-7 and 9-38, wherein the target gait parameters are input by the device wearer, the device manufacturer, a care provider, or a medical professional.

9. The ear-wearable device of any of claims 1-8 and 10-38, wherein the ear-wearable device is configured to calculate the one or more desired gait parameters by evaluating signals from the motion sensor and/or the microphone reflecting a current gait of the device wearer during an observation period.

10. The ear-wearable device of any of claims 1-9 and 11-38, wherein the ear-wearable device is configured to normalize the one or more desired gait parameters based on a detected activity level as reflected in data from the motion sensor.

11. The ear-wearable device of any of claims 1-10 and 12-38, wherein the ear-wearable device is configured to initiate or discontinue the series of audio cues based on a detected activity state as reflected in data from the motion sensor and/or the microphone.

12. The ear-wearable device of any of claims 1-11 and 13-38, the detected activity state comprising the device wearer assuming a standing or upright posture.

13. The ear-wearable device of any of claims 1-12 and 14-38, the detected activity state comprising cessation of the device wearer walking.

14. The ear-wearable device of any of claims 1-13 and 15-38, wherein the ear-wearable device is configured to initiate the series of audio cues based on detection of an abnormal or atypical gait.

15. The ear-wearable device of any of claims 1-14 and 16-38, wherein the ear-wearable device is configured to initiate the series of audio cues based on detection of a gait with a step timing variability or statistics crossing a threshold value.

16. The ear-wearable device of any of claims 1-15 and 17-38, wherein the threshold value is set through evaluation of previous events related to the gait of the device wearer.

17. The ear-wearable device of any of claims 1-16 and 18-38, wherein the threshold value is set through evaluation of previous events including one or more of gait freezes, stumbles, falls, cessation of walking, or continued walking with appreciably the same gait metrics or improved.

18. The ear-wearable device of any of claims 1-17 and 19-38, wherein the ear-wearable device is configured to initiate the series of audio cues based on detection of a gait with a left-right symmetry variability or statistics crossing a threshold value.

19. The ear-wearable device of any of claims 1-18 and 20-38, wherein the ear-wearable device is configured to initiate the series of audio cues based on detection of the presence of a device wearer within a particular environment.

20. The ear-wearable device of any of claims 1-19 and 21-38, the particular environment comprising an outdoor environment.

21. The ear-wearable device of any of claims 1-20 and 22-38, the particular environment comprising an indoor environment.

22. The ear-wearable device of any of claims 1-21 and 23-38, wherein the ear-wearable device is configured to interface with an accessory device and send or receive information regarding the one or more desired gait parameters.

23. The ear-wearable device of any of claims 1-22 and 24-38, wherein the ear-wearable device is configured to evaluate data from the motion sensor and/or microphone over a time period to determine a range of gait tempo values for the device wearer.

24. The ear-wearable device of any of claims 1-23 and 25-38, wherein the ear-wearable device is configured to set an audio property related to the series of audio cues based on a hearing loss of the device wearer.

25. The ear-wearable device of any of claims 1-24 and 26-38, the audio property comprising a volume, frequency specific amplification, frequency shifting, frequency compression, frequency transposition, and noise cancelation.

26. The ear-wearable device of any of claims 1-25 and 27-38, wherein the ear-wearable device is configured to adjust an audio property related to the series of audio cues based at least in part on an audiogram or other hearing test of the device wearer.

27. The ear-wearable device of any of claims 1-26 and 28-38, wherein the ear-wearable device is configured to evaluate a response of the device wearer in response to the series of audio cues.

28. The ear-wearable device of any of claims 1-27 and 29-38, wherein the ear-wearable device is configured to evaluate a response of the device wearer in response to the series of audio cues and adjust the series of audio cues accordingly.

29. The ear-wearable device of any of claims 1-28 and 30-38, wherein the ear-wearable device is configured to distinguish between a right step and a left step based on an input received from an accessory device.

30. The ear-wearable device of any of claims 1-29 and 31-38, wherein the ear-wearable device is configured to distinguish between a right step and a left step based on a signal from the motion sensor.

31. The ear-wearable device of any of claims 1-30 and 32-38, wherein the ear-wearable device is configured to characterize a gait of the device wearer at varying levels of physical exertion.

32. The ear-wearable device of any of claims 1-31 and 33-38, wherein the ear-wearable device is configured to characterize a gait of the device wearer at varying levels of cognitive exertion.

33. The ear-wearable device of any of claims 1-32 and 34-38, wherein the ear-wearable device is configured to detect whether the device wearer is changing elevation and changing a tempo of the series of audio cues accordingly.

34. The ear-wearable device of any of claims 1-33 and 35-38, the changing elevation comprising going up or down stairs or walking up or down a hill.

35. The ear-wearable device of any of claims 1-34 and 36-38, wherein the ear-wearable device is configured to sense ambient conditions around the device wearer and change a tempo of the series of audio cues accordingly.

36. The ear-wearable device of any of claims 1-35 and 37-38, wherein the ear-wearable device is configured to record identifying wireless packets encountered and cross-reference gait against the recorded identifying wireless packets.

37. The ear-wearable device of any of claims 1-36 and 38, the identifying wireless packets comprising BLUETOOTH advertising packets.

38. The ear-wearable device of any of claims 1-37, wherein the ear-wearable device is configured to record third party voices encountered and cross-reference gait against the recorded third party voices.

39. An ear-wearable device comprising:
a control circuit;
a motion sensor, wherein the motion sensor is electrical communication with the control circuit;
a microphone, wherein the microphone is in electrical communication with the control circuit; and
an electroacoustic transducer, wherein the electroacoustic transducer is in electrical communication with the control circuit;
wherein the ear-wearable device is configured to
operate in a first mode, wherein the first mode includes evaluating signals from at least one of the motion sensor and the microphone to characterize a gait of a device wearer; and
operate in a second mode, wherein the second mode includes providing a series of audio cues to the device wearer.

40. The ear-wearable device of any of claims 39 and 41-68, wherein the series of audio cues are configured to reflect an idealized gait specific for the device wearer.

41. The ear-wearable device of any of claims 39-40 and 42-68, wherein the idealized gait reflects one or more of an idealized gait tempo, gait cadence, step impact magnitude, and left vs. right symmetry value.

42. The ear-wearable device of any of claims 39-41 and 43-68, wherein the series of audio cues exhibit a left-right asymmetry that is characteristic for the device wearer.

43. The ear-wearable device of any of claims 39-42 and 44-68, wherein the series of audio cues comprise a rhythmic sequence.

44. The ear-wearable device of any of claims 39-43 and 45-68, wherein the series of audio cues comprise speech.

45. The ear-wearable device of any of claims 39-44 and 46-68, wherein the series of audio cues comprise virtual spatialized audio.

46. The ear-wearable device of any of claims 39-45 and 47-68, wherein the ear-wearable device is evaluate signals from at least one of the motion sensor and the microphone to detect whether the device wearer is walking.

47. The ear-wearable device of any of claims 39-46 and 48-68, wherein the ear-wearable device is configured to initiate or discontinue the series of audio cues based on a detected activity state as reflected in data from the motion sensor and/or the microphone.

48. The ear-wearable device of any of claims 39-47 and 49-68, the detected activity state comprising the device wearer assuming a standing or upright posture.

49. The ear-wearable device of any of claims 39-48 and 50-68, the detected activity state comprising cessation of the device wearer walking.

50. The ear-wearable device of any of claims 39-49 and 51-68, wherein the ear-wearable device is configured to initiate the series of audio cues based on detection of an abnormal or atypical gait.

51. The ear-wearable device of any of claims 39-50 and 52-68, wherein the ear-wearable device is configured to initiate the series of audio cues based on detection of a gait with a step timing variability or statistics crossing a threshold value.

52. The ear-wearable device of any of claims 39-51 and 53-68, wherein the threshold value is set through evaluation of previous events related to the gait of the device wearer.

53. The ear-wearable device of any of claims 39-52 and 54-68, wherein the ear-wearable device is configured to initiate the series of audio cues based on detection of a gait with a left-right symmetry variability or statistics crossing a threshold value.

54. The ear-wearable device of any of claims 39-53 and 55-68, wherein the threshold value is set through evaluation of previous events related to the gait of the device wearer.

55. The ear-wearable device of any of claims 39-54 and 56-68, wherein the ear-wearable device is configured to initiate the series of audio cues based on detection of the presence of the device wearer within a particular environment.

56. The ear-wearable device of any of claims 39-55 and 57-68, the particular environment comprising an outdoor environment.

57. The ear-wearable device of any of claims 39-56 and 58-68, the particular environment comprising an indoor environment.

58. The ear-wearable device of any of claims 39-57 and 59-68, wherein the ear-wearable device, when operating in the first mode, is configured to evaluate data from the motion sensor over a time period to determine a range of gait tempo values for the device wearer.

59. The ear-wearable device of any of claims 39-58 and 60-68, wherein the ear-wearable device is configured to evaluate a response of the device wearer in response to the series of audio cues.

60. The ear-wearable device of any of claims 39-59 and 61-68, wherein the ear-wearable device is configured to evaluate a response of the device wearer in response to the series of audio cues and adjust the series of audio cues accordingly.

61. The ear-wearable device of any of claims 39-60 and 62-68, wherein the ear-wearable device is configured to distinguish between a right step and a left step based on an input received from an accessory device.

62. The ear-wearable device of any of claims 39-61 and 63-68, wherein the ear-wearable device is configured to match the series of audio cues to the characterized gait of the device wearer.

63. The ear-wearable device of any of claims 39-62 and 64-68, wherein the ear-wearable device is configured to prompt the device wearer to execute specific actions while operating in the first mode.

64. The ear-wearable device of any of claims 39-63 and 65-68, the specific actions comprising a movement protocol.

65. The ear-wearable device of any of claims 39-64 and 66-68, wherein the specific actions are performed while the device wearer's eyes are closed.

66. The ear-wearable device of any of claims 39-65 and 67-68, wherein the ear-wearable device is configured to set an audio property related to the series of audio cues based on a hearing loss of the device wearer.

67. The ear-wearable device of any of claims 39-66 and 68, the audio property comprising a volume, frequency specific amplification, frequency shifting, frequency compression, frequency transposition, and noise cancelation.

68. The ear-wearable device of any of claims 39-67, wherein the ear-wearable device is configured to adjust an audio property related to the series of audio cues based at least in part on an audiogram or other hearing test of the device wearer.

69. An ear-wearable device comprising:
a control circuit;
a motion sensor, wherein the motion sensor is electrical communication with the control circuit;
a microphone, wherein the microphone is in electrical communication with the control circuit; and
an electroacoustic transducer, wherein the electroacoustic transducer is in electrical communication with the control circuit;
wherein the ear-wearable device is configured to
generate a set of data reflecting a current gait of a device wearer based on signals from at least one of the motion sensor and the microphone; and
match the set of data against a plurality of predetermined patterns to characterize the current gait.

70. The ear-wearable device of any of claims 69 and 71-77, wherein the ear-wearable device is configured to match the set of data against a plurality of predetermined patterns to characterize a current health status of the device wearer.

71. The ear-wearable device of any of claims 69-70 and 72-77, wherein the ear-wearable device is configured to determine whether the characterized current gait reflects a musculoskeletal injury or imbalance.

72. The ear-wearable device of any of claims 69-71 and 73-77, wherein the ear-wearable device is configured to alert the device wearer and/or a third party regarding the musculoskeletal injury or imbalance.

73. The ear-wearable device of any of claims 69-72 and 74-77, wherein the ear-wearable device is configured to generate a suggestion regarding a physical activity to ameliorate the musculoskeletal injury or imbalance.

74. The ear-wearable device of any of claims 69-73 and 75-77, wherein the ear-wearable device is configured to identify whether the device wearer is using a walking assistance device.

75. The ear-wearable device of any of claims 69-74 and 76-77, the walking assistance device comprising a cane, a walker, a knee walker, or crutches.

76. The ear-wearable device of any of claims 69-75 and 77, wherein the ear-wearable device is configured to identify a condition of hypokinetic feet based on the characterization of the current gait.

77. The ear-wearable device of any of claims 69-76, wherein the ear-wearable device is configured to characterize a current emotional status of the device wearer based on the current gait of the device wearer.

78. An ear-wearable device comprising:
a control circuit;
a motion sensor, wherein the motion sensor is in electrical communication with the control circuit;
a microphone, wherein the microphone is in electrical communication with the control circuit; and
an electroacoustic transducer, wherein the electroacoustic transducer is in electrical communication with the control circuit;
wherein the ear-wearable device is configured to
generate a set of data reflecting a current gait of a device wearer based on signals from at least one of the motion sensor and the microphone;
compare the set of data against stored data reflecting a previous gait of the device wearer; and
characterize a health status of the device wearer based on a change from the previous gait to the current gait of the device wearer.

79. The ear-wearable device of any of claims 78 and 80-90, wherein the ear-wearable device is configured to determine whether the change from the previous gait to the current gait reflects an injury.

80. The ear-wearable device of any of claims 78-79 and 81-90, wherein the ear-wearable device is configured to determine whether the change from the previous gait to the current gait reflects a neurological disease state or a neurological injury.

81. The ear-wearable device of any of claims 78-80 and 82-90, wherein the ear-wearable device is configured to determine whether the change from the previous gait to the current gait reflects an elevated fall risk.

82. The ear-wearable device of any of claims 78-81 and 83-90, wherein the ear-wearable device is configured to initiate audio cues for delivery to the device wearer when an elevated fall risk is present.

83. The ear-wearable device of any of claims 78-82 and 84-90, wherein the device wearer is configured to send a control signal to a secondary device when an elevated fall risk is present.

84. The ear-wearable device of any of claims 78-83 and 85-90, the secondary device comprising a home automation device.

85. The ear-wearable device of any of claims 78-84 and 86-90, wherein the ear-wearable device is configured to determine whether the change from the previous gait to the current gait reflects a reduced fall risk.

86. The ear-wearable device of any of claims 78-85 and 87-90, wherein the ear-wearable device is configured to determine whether the change from the previous gait to the current gait reflects an improved health state.

87. The ear-wearable device of any of claims 78-86 and 88-90, wherein the ear-wearable device is configured to send a notification of the health status of the device wearer to a third party.

88. The ear-wearable device of any of claims 78-87 and 89-90, wherein the ear-wearable device is configured to determine whether the change from the previous gait to the current gait reflects a slowing gait tempo.

89. The ear-wearable device of any of claims 78-88 and 90, wherein the ear-wearable device is configured to cross-reference changes in gait with changes in activity levels of the device wearer.

90. The ear-wearable device of any of claims 78-89, wherein the ear-wearable device is configured to cross-reference changes in gait with changes in footwear of the device wearer as identified by at least one of signals from the microphone and input from the device wearer.

91. An ear-wearable system comprising:
a first ear-wearable device, the first ear-wearable device comprising
a first control circuit;
a first motion sensor, wherein the first motion sensor is in electrical communication with the first control circuit;
a first microphone, wherein the first microphone is in electrical communication with the first control circuit; and
a first electroacoustic transducer, wherein the first electro-acoustic transducer is in electrical communication with the first control circuit;
a second ear-wearable device, the second ear-wearable device comprising
a second control circuit;
a second motion sensor, wherein the second motion sensor is in electrical communication with the second control circuit;
a second microphone, wherein the second microphone is in electrical communication with the second control circuit; and
a second electroacoustic transducer, wherein the second electro-acoustic transducer is in electrical communication with the second control circuit;
wherein the ear-wearable system is configured to
calculate one or more desired gait parameters; and
provide a series of audio cues to a device wearer consistent with the one or more desired gait parameters.

92. The ear-wearable system of any of claims 91 and 93-95, wherein the series of audio cues are delivered differentially through the first ear-wearable device and the second ear-wearable device.

93. The ear-wearable system of any of claims 91-92 and 94-95, wherein the ear-wearable system is configured to compare signals from the first ear-wearable device and the second ear-wearable device to discriminate between a right side footfall and a left side footfall.

94. The ear-wearable system of any of claims 91-93 and 95, wherein the ear-wearable system is configured to duty cycle operations between the first ear-wearable device and the second ear-wearable device.

95. The ear-wearable system of any of claims 91-94, the series of audio cues comprising virtual spatialized audio.
